(19) United States
(12) Patent Application Publication
(10) Pub. No.: US 2025/0084167 A1
(43) Pub. Date: Mar. 13, 2025

(54) METHODS AND COMPOSITIONS FOR TREATING IgG4-RELATED DISEASES

(71) Applicant: Zenas BioPharma, Inc., Waltham, MA (US)

(72) Inventors: Allen POMA, Somerville, MA (US); Shauna QUINN, New York, NY (US); Xiaodong WANG, Somerville, MA (US)

(73) Assignee: Zenas BioPharma, Inc., Waltham, MA (US)

(21) Appl. No.: 18/960,561

(22) Filed: Nov. 26, 2024

Related U.S. Application Data
(63) Continuation of application No. PCT/US2023/067849, filed on Jun. 2, 2023.
(60) Provisional application No. 63/348,861, filed on Jun. 3, 2022.
(60) Provisional application No. 63/386,781, filed on Dec. 9, 2022.
(60) Provisional application No. 63/497,929, filed on Apr. 24, 2023.

(51) Int. Cl. C07K 16/28; A61K 39/00; A61P 37/06

(52) U.S. Cl. CPC C07K 16/2803; A61P 37/06; A61K 2039/54; A61K 2039/545

(57) ABSTRACT

The present invention provides compositions and methods of treating and improving the symptoms of IgG4-RD using an antibody or antigen-binding fragment thereof that specifically binds human CD19.

### CROSS-REFERENCE TO RELATED APPLICATIONS

This application is a continuation of International Application No. PCT/US2023/067849, filed Jun. 2, 2023, which claims priority to U.S. Provisional Application No. 63/348,861, filed Jun. 3, 2022, U.S. Provisional Application No. 63/386,781, filed Dec. 9, 2022, and U.S. Provisional Application No. 63/497,929, filed Apr. 24, 2023, each of which are hereby incorporated by reference in their entireties.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Nov. 22, 2024, is named “ZEN-007US1.XML” and is 13,225 bytes in size.

### BACKGROUND

IgG4-Related Disease (IgG4-RD) is a serious, rare, chronic fibro-inflammatory condition that typically affects multiple organs, leading to significant organ dysfunction and failure, and even death if the condition is not recognized and treated in a timely manner. The disease commonly affects major organs such as the pancreas, liver, kidneys, lungs, and eyes; pancreato-hepatobiliary disease is present in approximately half of cases (Wallace et al, 2019). Patients may present with a single organ involved but more frequently present with multiple organ involvement. As the disease progresses and patients experience new or worsening signs/symptoms (flares), additional organs develop lesions, and the cellular inflammation characterizing early disease moves towards a more fibrotic stage, causing major tissue damage, and ultimately organ failure (Perugino 2016). For example, cholangitis due to IgG4-RD can lead to hepatic failure, IgG4-related (type 1) autoimmune pancreatitis can lead to failure of either or both the endocrine and exocrine pancreas, and IgG4-related aortitis can lead to aneurysms and/or aortic dissection. Current therapies used for long term treatment of IgG4-RD frequently cause significant toxicity and must be discontinued. There is an unmet need for patients with active IgG4-RD.

### SUMMARY

The present disclosure provides both improving treatment of a human patient with active (on-going flare) IgG4-RD and the prevention of relapse (recurrence of flare) of IgG4-RD, using an CD19 antibody (e.g., obexelimab). The present disclosure is directed to improved treatment of a human patient with active IgG-RD and prevention of relapse of IgG4-RD using a CD19 antibody (e.g., obexelimab). The present disclosure is also directed to methods of preventing relapse of IgG4-RD using a CD19 antibody (e.g., obexelimab). Among other things, this disclosure provides methods, compositions and use of a CD19 antibody (e.g., obexelimab) in adult human patients (i.e., 18 years of age or older) with IgG4-RD, including those with new, relapsed or refractory IgG4-RD, to treat, improve, stabilize or reduce one or more symptoms of IgG4-RD, such as reducing incidence of disease flare.

In some aspects, this disclosure provides methods, compositions and use of a CD19 antibody (e.g., obexelimab) in adult human patients (i.e., 18 years of age or older) with IgG4-RD, including those with new, relapsed or refractory IgG4-RD, to treat, improve, stabilize or reduce one or more symptoms of IgG4-RD, such as reducing incidence or severity of disease flare.

In one aspect, the present invention provides a method of treating or preventing IgG4-related disease (IgG4-RD), comprising administering obexelimab subcutaneously to a human patient ≥18 years of age at a dose of 250 mg per week. In one aspect, the present invention provides a method of treating IgG4-related disease (IgG4-RD), comprising administering obexelimab subcutaneously to a human patient ≥18 years of age at a dose of 250 mg once a week.

In some embodiments, the method comprises administering obexelimab subcutaneously at a dose of 125 mg twice a week. In some embodiments, the method comprises administering obexelimab subcutaneously at a dose of 125 mg twice a week. In some embodiments, the method comprises administering obexelimab subcutaneously at a dose of 125 mg every 3 days. In some embodiments, the method comprises administering obexelimab subcutaneously at a dose of 250 mg every 7 days.

In some embodiments, obexelimab can be administered subcutaneously to a human patient ≥18 years of age at a dose of 200 mg once a week. In some embodiments, obexelimab can be administered subcutaneously to a human patient 18 years of age at a dose of 125 mg twice a week. In some embodiments, obexelimab is administered subcutaneously to a human patient ≥18 years of age at a dose of 100 mg twice a week. In some embodiments, obexelimab is administered subcutaneously to a human patient 18 years of age at a dose of 300 mg once a week. In some embodiments, obexelimab is administered subcutaneously to a human patient ≥18 years of age at a dose of 150 mg twice a week.

In some embodiments, obexelimab is administered in a liquid formulation comprising 125 mg/mL obexelimab. In some embodiments, obexelimab is administered as 2×1 mL injections or 1×2 mL injection. In some embodiments, obexelimab is administered as 2×1 mL injections or 1×2 mL injection for a total dose of 250 mg. In some embodiments, obexelimab injections are administered concurrently. In some embodiments, obexelimab injections are administered within 1 minute, 2 minutes, 3 minutes, 4 minutes, 5 minutes, 6 minutes, 7 minutes, 8 minutes, 9 minutes, 10 minutes, or within 20 minutes of each other. In some embodiments, obexelimab injections are administered within 1 hour, within 2 hours, within 3 hours, within 4 hours, within 5 hours, within 6 hours, within 7 hours, within 8 hours, within 9 hours, within 10 hours, within 11 hours, or within 12 hours of each other.

In some embodiments, obexelimab is administered in a liquid formulation comprising 125 mg/mL obexelimab as 4×0.25 mL injections. In some embodiments, obexelimab is administered in a liquid formulation comprising 125 mg/mL obexelimab as 4×0.25 mL injections for a total dose of 250 mg. In some embodiments, obexelimab injections are administered concurrently. In some embodiments, obexelimab injections are administered within 1 minute, 2 minutes, 3 minutes, 4 minutes, 5 minutes, 6 minutes, 7 minutes, 8 minutes, 9 minutes, 10 minutes, or within 20 minutes of each other. In some embodiments, obexelimab injections are administered within 1 hour, within 2 hours, within 3 hours, within 4 hours, within 5 hours, within 6 hours, within 7 hours, within 8 hours, within 9 hours, within 10 hours, within 11 hours, or within 12 hours of each other.

In some embodiments, obexelimab is administered in a liquid formulation as a single injection. In some embodiments, obexelimab is administered in a liquid formulation as a single injection for a total dose of 250 mg.

In some embodiments, obexelimab is self-injected by the patient. In some embodiments, obexelimab is injected by a person who is not the patient (e.g., a caregiver). In some embodiments, obexelimab is administered under the supervision of a clinician.

In some embodiments, the obexelimab is administered in a liquid formulation comprising 125 mg/mL obexelimab, 2.35 mg/mL sodium acetate trihydrate, 0.17 mg/mL acetic acid, 30 mg/mL L-proline, 0.1 mg/mL polysorbate 80 at pH 5.5. In some embodiments, the obexelimab is administered in a liquid formulation comprising 100 mg/mL obexelimab, 2.35 mg/mL sodium acetate trihydrate, 0.17 mg/mL acetic acid, 30 mg/mL L-proline, 0.1 mg/mL polysorbate 80 at pH 5.5. In some embodiments, the anti-CD19 antibody (e.g., obexelimab) is formulated at a concentration of 80-200 mg/mL obexelimab, 1.5-3 mg/mL sodium acetate trihydrate, 0.10-0.20 mg/mL acetic acid (at density 1.053 g/mL), 10-50 mg/mL L-proline, 0.05-0.2 mg/mL polysorbate 80, pH 5.0-6.0.

In some embodiments, the method encompasses administration of the anti-CD19 antibody (e.g., obexelimab) to a human patient that has received glucocorticoid (GC) therapy. In some embodiments, the method encompasses administration of the anti-CD19 antibody (e.g., obexelimab) to a human patient that has not received glucocorticoid (GC) therapy.

In some embodiments, the GC therapy is administered at a dose of 20-60 mg/day prednisone or equivalent. In some embodiments, the GC therapy is administered at a dose of 10-100 mg/day prednisone or equivalent. In some embodiments, the GC therapy is administered at a dose of about 1-70 mg/day, about 5-70 mg/day, about 10-70 mg/day, about 15-70 mg/day, about 20-70 mg/day, about 25-70 mg/day, about 30-70 mg/day, about 35-70 mg/day, about 40-70 mg/day prednisone or equivalent. In some embodiments, the GC therapy is administered at a dose of about 1-60 mg/day, about 5-60 mg/day, about 10-60 mg/day, about 15-60 mg/day, about 20-60 mg/day, about 25-60 mg/day, about 30-60 mg/day, about 35-60 mg/day, about 40-60 mg/day prednisone or equivalent.

In some embodiments, the GC therapy is administered at a dose of about 1-150 mg/day, about 5-150 mg/day, about 10-150 mg/day, about 15-150 mg/day, about 20-150 mg/day, about 25-150 mg/day, about 30-150 mg/day, about 35-150 mg/day, about 40-150 mg/day, about 45-150 mg/day, about 50-150 mg/day, about 55-150 mg/day, about 60-150 mg/day, about 65-150 mg/day, about 70-150 mg/day, about 75-150 mg/day, about 80-150 mg/day, about 90-150 mg/day, or about 100-150 mg/day, prednisone or equivalent. In some embodiments, the GC therapy is administered at a dose of about 5-120 mg/day, about 5-110 mg/day, about 10-90 mg/day, about 15-100 mg/day, about 20-100 mg/day, about 25-100 mg/day, about 30-100 mg/day, about 35-100 mg/day, about 40-100 mg/day, about 45-100 mg/day, about 50-100 mg/day, about 55-100 mg/day, about 60-100 mg/day, about 65-100 mg/day, about 70-100 mg/day, about 75-100 mg/day, about 80-100 mg/day, or about 90-100 mg/day prednisone or equivalent.

In some embodiments, the GC therapy is administered at a dose of up to about 150 mg/day, up to about 120 mg/day, up to about 110 mg/day, up to about 100 mg/day, up to about 90 mg/day, up to about 80 mg/day, up to about 70 mg/day, up to about 60 mg/day, up to about 50 mg/day, up to about 40 mg/day, up to about 30 mg/day, up to about 20 mg/day, up to about 15 mg/day, up to about 10 mg/day, up to about 5 mg/day, or up to about 1 mg/day prednisone or equivalent.

In some embodiments, the GC therapy is administered at a dose of 0.1-1 mg/kg/day, a dose of 0.1-0.8 mg/kg/day, a dose of 0.1-0.7 mg/kg/day, a dose of 0.1-0.6 mg/kg/day, a dose of 0.1-0.5 mg/kg/day, a dose of 0.1-0.4 mg/kg/day, a dose of 0.1-0.3 mg/kg/day, a dose of 0.1-0.2 mg/kg/day or a dose of 0.05-0.1 mg/kg/day prednisone or equivalent.

In some embodiments, the GC therapy is administered at a dose of up to 1 mg/kg/day prednisone or equivalent. In some embodiments, the GC therapy is administered at a dose of up to 0.9 mg/kg/day, a dose of up to 0.8 mg/kg/day, a dose of up to 0.7 mg/kg/day, a dose of up to 0.6 mg/kg/day, a dose of up to 0.5 mg/kg/day, a dose of up to 0.4 mg/kg/day, a dose of up to 0.3 mg/kg/day, a dose of up to 0.2 mg/kg/day or dose of up to 0.1 mg/kg/day prednisone or equivalent.

In some embodiments, the prednisone equivalent doses are determined as shown in Table 3.

In some embodiments, the GC therapy is administered at a high dose of prednisone or equivalent. In some embodiments, the patient has been administered a high dose of GC therapy and is unlikely to respond to any treatment therapy. In some embodiments, the patient has not responded to prior therapies before administration of the anti-CD19 antibody (e.g., obexelimab).

In some embodiments, the GC therapy continues during the treatment with obexelimab. In some embodiments, the GC therapy is tapered during treatment with obexelimab. In some embodiments, the GC therapy is tapered prior to treatment with obexelimab. In some embodiments, the GC therapy is tapered to complete discontinuation. In some embodiments, obexelimab is administered in combination with a GC therapy. In some embodiments, obexelimab is administered without the use of GC therapy.

In some embodiments, obexelimab is administered for a time period sufficient to treat, improve, stabilize or reduce one or more symptoms of IgG4-RD relative to a control. In some embodiments, at least one symptom is exhibited in an organ selected from lymph nodes, submandibular glands, parotid glands, lacrimal glands, kidney, heart, pericardium, orbit, nasal cavity, lungs, bile ducts, salivary glands, and pancreas.

In some embodiments, obexelimab is administered to the patient until complete remission. In some embodiments, obexelimab is administered to the human patient during a recurrence-free period.

In some embodiments, obexelimab is administered to the patient until complete remission or until a physician considers the patient clinically without evidence of disease flare. In some embodiments, obexelimab is administered until a physician considers the patient clinically without evidence of disease flare. In some embodiments, obexelimab is administered to the patient until the patient is clinically without evidence of disease flare.

In some embodiments, the recurrence-free period is referred to as time to disease flare (TDF). In some embodiments, the TDF is measured in days. In some embodiments, the TDF is measured in weeks. In some embodiments, the TDF is measured in months. In some embodiments, the TDF is measured in years.

In some embodiments, the recurrence-free period is determined by the days to disease flare. In some embodiments, the recurrence-free period is at least 10 days, 20 days, 30 days, 40 days, 50 days, 60 days, 70 days, 80 days, or 90 days from the first administration of obexelimab. In some embodiments, the recurrence-free period is at least 28 days, at least 35 days, at least 42 days, at least 49 days, at least 56 days, at least 63 days, at least 70 days, at least 77 days, at least 84 days, at least 91 days, at least 98 days, at least 105 days, at least 112 days, at least 119 days, at least 126 days, at least 133 days, at least 140 days, at least 147 days, at least 154 days, at least 161 days, at least 168 days, at least 175 days, at least 182 days, at least 189 days, at least 196 days, at least 203 days, at least 210 days, at least 217 days, at least 224 days, at least 231 days, at least 238 days, at least 245 days, at least 252 days, at least 259 days, at least 266 days, at least 273 days, at least 280 days, 287 days, at least 294 days, at least 301 days, at least 308 days, at least 315 days, at least 322 days, at least 329 days, at least 336 days, at least 343 days, at least 350 days, at least 357 days, or at least 364 days from the first administration of obexelimab.

In some embodiments, the recurrence-free period is at least 60 days, at least 70 days, at least 80 days, at least 90 days, at least 100 days, at least 120 days, at least 150 days, at least 160 days, at least 190 days, at least 220 days, at least 250 days, at least 280 days, at least 310 days, at least 340 days, or at least 365 days from the first administration of obexelimab. In some embodiments, the recurrence-free period is at least 160 days. In some embodiments, the recurrence-free period is at least 160 days, at least 161 days, at least 162 days, at least 163 days, at least 164 days, at least 165 days, at least 166 days, at least 167 days, at least 168 days, at least 169 days, at least 170 days, at least 171 days, at least 172 days, at least 173 days, at least 174 days, at least 175 days, at least 176 days, at least 177 days, at least 178 days, at least 179 days, or at least 180 days. In some embodiments, the recurrence-free period is at least 169 days from the first administration of obexelimab.

In some embodiments, the recurrence-free period is at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, or at least 43 weeks, from the first administration of obexelimab. In some embodiments, the recurrence-free period is at least 52 weeks from the first administration of obexelimab. In some embodiments, the recurrence-free period is at least 40 weeks, at least 45 weeks, at least 50 weeks, at least 52 weeks, at least 55 weeks, at least 60 weeks from the administration of obexelimab. In some embodiments, the recurrence-free period is at least 52 weeks from administration of obexelimab.

In some embodiments, the recurrence-free period is up to 40 weeks, up to 45 weeks, up to 50 weeks, up to 52 weeks, up to 55 weeks, up to 60 weeks from the first administration of obexelimab. In some embodiments, the recurrence-free period is up to 52 weeks from the first administration of obexelimab.

In some embodiments, the recurrence-free period is at least 2 months from the first administration of obexelimab. In some embodiments, the recurrence-free period is at least 6 months from the first administration of obexelimab. In some embodiments, the recurrence-free period is at least 9 months from the first administration of obexelimab. In some embodiments, the recurrence-free period is at least 12 months from the first administration of obexelimab. In some embodiments, the recurrence-free period is at least 18 months from the first administration of obexelimab. In some embodiments, the recurrence-free period is at least 24 months from the first administration of obexelimab. In some embodiments, the recurrence-free period is at least 30 months from the first administration of obexelimab.

In some embodiments, the recurrence-free period is at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, at least 24 months, at least 25 months, at least 26 months, at least 27 months, at least 28 months, at least 29 months or at least 30 months from the first administration of obexelimab.

In some embodiments, the recurrence-free period is at least 2-12 months, at least 2-10 months, at least 2-8 months, at least 2-6 months, or at least 2-4 months from the first administration of obexelimab. In some embodiments, the recurrence-free period is at least 3-12 months, at least 3-10 months, at least 3-8 months, at least 3-6 months, at least 3-5 months, or at least 3-4 months from the first administration of obexelimab. In some embodiments, the recurrence-free period is at least 4-12 months, at least 4-10 months, at least 4-8 months, at least 4-6 months, or at least 4-5 months from the first administration of obexelimab. In some embodiments, the recurrence-free period is at least 5-12 months, at least 5-10 months, at least 5-8 months, or at least 5-6 months from the first administration of obexelimab. In some embodiments, the recurrence-free period is at least 6-12 months, at least 6-10 months, at least 6-8 months, or at least 6-7 months from the first administration of obexelimab.

In some embodiments, the recurrence-free period is at least 1 year, least 1.5 years, at least 2 years, at least 2.5 years, at least 3 years, at least 4 years, or at least 5 years, from the first administration of obexelimab.

In some embodiments, obexelimab is administered as a maintenance therapy. In some embodiments, the patient is recurrence-free while taking obexelimab. In some embodiments, the patient is recurrence-free while taking obexelimab at a dose of 250 mg once a week. In some embodiments, the recurrence-free period is until obexelimab is discontinued. In some embodiments, the recurrence-free period at least 7 days after obexelimab is discontinued. In some embodiments, the recurrence-free period at least 14 days after obexelimab is discontinued. In some embodiments, the recurrence-free period at least 21 days after obexelimab is discontinued. In some embodiments, the recurrence-free period at least 28 days after obexelimab is discontinued.

In some embodiments, obexelimab is administered to the patient until the patient has active IgG4-RD disease flare. In some embodiments, obexelimab is administered to the patient until the patient has active IgG4-RD flare that requires initiation of rescue therapy. In some embodiments, obexelimab is administered while the patient has active IgG4-RD disease flare.

In some embodiments, the rescue therapy comprises administration of GC therapy. In some embodiments, rescue therapy involves initiation of a non-GC therapy, which control the symptoms of flare.

In some embodiments, the human patient is relapsed or refractory to a previous treatment for IgG4-RD. In some embodiments, the human patient is relapsed or refractory to rituximab. In some embodiments, the human patient is relapsed or refractory to another CD19 therapy.

In some embodiments, the human patient is assessed for disease activity using the IgG4-RD responder index (RI). In some embodiments, the human patient achieves a ≥2 points decrease in IgG4-RD RI from day 1 of administration with obexelimab. In some embodiments, the human patient has met the American College of Rheumatology (ACR)/European League Against Rheumatism (EULAR) classification criteria for IgG4-RD score (IgG4-RD score) >20 prior to administration of obexelimab.

In some embodiments, obexelimab is administered for a time period sufficient to improve, stabilize or reduce one or more symptoms of IgG4-RD relative to a control. In some embodiments, at least one symptom is exhibited in an organ selected from lymph nodes, submandibular glands, parotid glands, lacrimal glands, kidney, heart, pericardium, orbit, nasal cavity, lungs, bile ducts, salivary glands, and pancreas. In some embodiments, obexelimab is administered to the patient until complete remission. In some embodiments, obexelimab is administered to the patient at an administrative interval during a recurrence-free period. In some embodiments, the recurrence-free period is determined by the days to disease flare. In some embodiments, the recurrence-free period is at least 30 days, 40 days, 50 days, 60 days, 70 days, 80 days, or 90 days from the first administration of obexelimab. In some embodiments, the recurrence-free period is more than 90 days. In some embodiments, the recurrence-free period is up to 40 weeks, up to 45 weeks, up to 50 weeks, up to 52 weeks from the first administration of obexelimab.

In some embodiments, obexelimab is administered to the patient until the patient has active IgG4-RD flare that requires initiation of rescue therapy. In some embodiments, the rescue therapy comprises administration of GC therapy. In some embodiments, the rescue therapy comprises administration of GC therapy in combination with obexelimab. In some embodiments, the patient is relapsed or refractory to a previous treatment for IgG4-RD. In some embodiments, the patient is relapsed or refractory to rituximab.

In some embodiments, the patient presents with an IgG4-RD manifestation selected from the group consisting of IgG4-related sialadenitis (chronic sclerosing sialadenitis, Kuttner's tumour, Mikulicz's disease), IgG4-related dacryoadenitis (Mikulicz's disease), IgG4-related ophthalmic disease (idiopathic orbital inflammatory disease, orbital pseudotumor), chronic sinusitis IgG4-related hypophysitis (IgG4-related panhypophysitis, IgG4-related adenohypophysitis, IgG4-related infundibuloneurohypophysitis, autoimmune hypophysitis), IgG4-related pachymeningitis, IgG4-related leptomeningitis (idiopathic hypertrophic pachymeningitis), IgG4-related pancreatitis (Type 1 autoimmune pancreatitis, IgG4-related AIP, lymphoplasmacytic sclerosing pancreatitis, chronic pancreatitis with diffuse irregular narrowing of the main pancreatic duct), IgG4-related lung disease (Pulmonary inflammatory pseudotumour), IgG4-related pleuritis, IgG4-related hepatopathy, IgG4-related sclerosing cholangitis, IgG4-related cholecystitis, IgG4-related aortitis (inflammatory aortic aneurysm), IgG4-related periaortitis (chronic periaortitis), IgG4-related periarteritis, IgG4-related pericarditis, IgG4-related mediastinitis (fibrosing mediastinitis), IgG4-related retroperitoneal fibrosis (retroperitoneal fibrosis, Albarran-Ormond syndrome, Ormond's disease (retroperitoneal fibrosis), perirenal fasciitis, Gerota's fasciitis/syndrome, periureteritis fibrosa, sclerosing lipogranuloma, sclerosing retroperitoneal granuloma, non-specific retroperitoneal inflammation, sclerosing retroperitonitis, retroperitoneal vasculitis with perivascular fibrosis), IgG4-related mesenteritis (subtypes are: mesenteric panniculitis, mesenteric lipodystrophy and retractile mesenteritis) (sclerosing mesenteritis, systemic nodular panniculitis, liposclerosis mesenteritis, mesenteric Weber-Christian disease, mesenteric lipogranuloma, xanthogranulomatous mesenteritis), IgG4-related mastitis (sclerosing mastitis), IgG4-related kidney disease (IgG4-RKD), IgG4-related tubulointerstitial nephritis (IgG4-TIN), IgG4-related membranous glomerulonephritis (idiopathic tubulointerstitial nephritis), IgG4-related prostatitis, IgG4-related perivasal fibrosis (chronic orchialgia), IgG4-related paratesticular pseudotumor, IgG4-related epididymo-orchitis (paratesticular fibrous pseudotumor, inflammatory pseudotumor of the spermatic cord, pseudosarcomatous myofibroblastic proliferations of the spermatic cord, proliferative funiculitis, chronic proliferative periorchitis, fibromatous periorchitis, nodular periorchitis, reactive periorchitis, fibrous mesothelioma), IgG4-related lymphadenopathy, IgG4-related skin disease (angiolymphoid hyperplasia with eosinophilia, cutaneous pseudolymphoma), IgG4-related perineural disease, and IgG4-related thyroid disease (Reidel's thyroiditis), inflammatory pseudotumour, and multifocal fibrosclerosis.

In some embodiments, the patient presents with a an IgG4-RD manifestation selected from the group consisting of autoimmune pancreatitis (lymphoplasmacytic scleorising pancreatitis), eosinophilic angiocentric fibrosis (affecting the orbits and upper respiratory tract), fibrosing mediastinitis, idiopathic hypertrophic pachymeningitis, idiopathic tubulointerstitial nephritis, inflammatory pseudotumour, Kuttner's tumour, Mikulicz's disease, fibrosclerosis, periaortitis, periarteritis, inflammatory aortic multifocal aneurysm, Ormond's disease (tetroperitoneal fibrosis), Riedel's thyroiditis, and sclerosing mesenteritis.

### BRIEF DESCRIPTION OF FIGURES

FIG. 1 is a graph showing PK simulation of Obexelimab Plasma Concentration-Time Profiles after Weekly Subcutaneous Administration of Obexelimab doses.

FIG. 2 is a graph showing simulation of Absolute B Cell Count Change from Baseline after Weekly Subcutaneous Administration of Obexelimab doses.

FIG. 3 is a graph showing simulations of CD19 Receptor Occupancy after Weekly Subcutaneous Administration of Obexelimab

### DETAILED DESCRIPTION

The present invention provides, among other things, methods of treating IgG4-related disease by administering to a human patient >18 years of age in need of treatment an anti-CD19 antibody at a therapeutically effective dose and an administration interval for a treatment period sufficient to improve, stabilize or reduce one or more symptoms of IgG4-related disease relative to a control (e.g., start of treatment).

In one aspect, the present invention provides a method of reducing the risk of flare recurrence in adult patients. In one aspect, the present invention provides a method of reducing the risk of flare recurrence in adult patients with IgG4-RD (immunoglobulin G4 related diseases) comprising administering obexelimab to the patient. In one aspect, the present invention provides a method of treating active IgG4-RD (immunoglobulin G4 related diseases) comprising administering obexelimab to the patient.

In one aspect, the present invention provides a method of preventing flare recurrence in adult patients. In one aspect, the present invention provides a method of preventing flare recurrence in adult patients with IgG4-RD (immunoglobulin G4 related diseases) comprising administering obexelimab to the patient. In one aspect, the present invention provides a method of preventing signs or symptoms of active IgG4-RD (immunoglobulin G4 related diseases) comprising administering obexelimab to the patient.

In some embodiments, the method comprises administering obexelimab at a dose of 250 mg. In some embodiments, the method comprises administering obexelimab at a dose of 250 mg every 7 days. In some embodiments, the method comprises administering obexelimab at a dose of 250 mg every 7 days to an adult patient (e.g., a patient at least 18 years old). In some embodiments, the method comprises administering obexelimab subcutaneously.

In one aspect, the present invention provides a method of reducing the risk of flare recurrence in adult patients comprising administering obexelimab subcutaneously at a dose of 250 mg every 7 days to an adult patient.

Various aspects of the invention are described in detail in the following sections. The use of sections is not meant to limit the invention. Each section can apply to any aspect of the invention. In this application, the use of “or” means “and/or” unless stated otherwise.

### Definitions

Described herein are several definitions. Such definitions are meant to encompass grammatical equivalents.

Antibody: The term “antibody” herein is meant to include a protein consisting of one or more polypeptides substantially encoded by all or part of the recognized immunoglobulin genes. The recognized immunoglobulin genes, for example in humans, include the kappa (κ), lambda (1), and heavy chain genetic loci, which together comprise the myriad variable region genes, and the constant region genes mu (u), delta (d), gamma (γ), sigma (s), and alpha (a) which encode the IgM, IgD, IgG (IgG1, IgG2, IgG3, and IgG4), IgE, and IgA (IgA1 and IgA2) isotypes respectively. Antibody herein is meant to include full length antibodies and antibody fragments, and may refer to a natural antibody from any organism, an engineered antibody, or an antibody generated recombinantly for experimental, therapeutic, or other purposes.

CD32b⁺ cell or FcγRIIb⁺ cell: The terms “CD32b⁺ cell” or “FcγRIIb⁺ cell” as used herein is meant any cell or cell type that expresses CD32b (FcγRIIb). CD32b+ cells include but are not limited to B cells, plasma cells, dendritic cells, macrophages, neutrophils, mast cells, basophils, or eosinophils.

CDC or complement dependent cytotoxicity: The terms “CDC” or “complement dependent cytotoxicity” as used herein is meant the reaction wherein one or more complement protein components recognize bound antibody on a target cell and subsequently cause lysis of the target cell.

Effector Function: The term “effector function” as used herein is meant a biochemical event that results from the interaction of an antibody Fc region with an Fc receptor or ligand. Effector functions include FcγR-mediated effector functions such as ADCC and ADCP, and complement-mediated effector functions such as CDC. Further, effector functions include FcγRIIb-mediated effector functions, such as inhibitory functions (e.g., downregulating, reducing, inhibiting etc., B cell responses, e.g., a humoral immune response).

Effector Cell: The term “effector cell” as used herein is meant a cell of the immune system that expresses one or more Fc and/or complement receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and gd T cells, and may be from any organism including but not limited to humans, mice, rats, rabbits, and monkeys.

Fc or Fc region: The terms “Fc” or “Fc region,” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cgamma2 and Cgamma3 (Og2 and C 3) and the hinge between Cgammal (Ogi) and Cgamma2 (Cy2). Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an Fc polypeptide, as described below.

Fc gamma receptor, or FcγR: The terms “Fc gamma receptor” or “FcγR” as used herein is meant any member of the family of proteins that bind the IgG antibody Fc region and are substantially encoded by the FcγR genes. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRI 11 (CD1 6), including isoforms FcγRI lla (including allotypes V1 58 and F158) and FcγRIIIb (including allotypes FcγRlllb-NA1 and FcγRI llb-NA2) (Jefferis et a/., 2002, Immunol Lett 82:57-65, incorporated entirely by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγR 111 (CD16), and FcγRIII-2 (CD16-2), as well as any undiscovered mouse FcγRs or FcγR isoforms or allotypes.

Flare: The term “flare”, in the context of IgG4-RD is defined as the reappearance of previous signs/symptoms or appearance of new signs/symptoms of IgG4-RD. In certain embodiments, “flare”, in the context of IgG4-RD is the reappearance of previous signs/symptoms or appearance of new signs/symptoms of IgG4-RD that, in the opinion of the investigator and the adjudication committee, require initiation of rescue therapy. In some embodiments, an IgG4-RD flare is the reappearance of previous signs/symptoms or appearance of new signs/symptoms of IgG4-RD that fulfill the IgG4-RD Flare Criteria.

Modification: The term “modification” herein is meant an alteration in the physical, chemical, or sequence properties of a protein, polypeptide, antibody, or immunoglobulin. Modifications described herein include amino acid modifications and glycoform modifications.

Target Antigen: The term “target antigen” as used herein is meant the molecule that is bound by the variable region of a given antibody, or the fusion partner of an Fc fusion. A target antigen may be a protein, carbohydrate, lipid, or other chemical compound. An antibody or Fc fusion is said to be “specific” for a given target antigen based on having affinity for the target antigen. In some embodiments, the target antigen for obexelimab is CD19.

Target cell: The term “target cell” as used herein is meant a cell that expresses a target antigen.

Obexelimab: The term “Obexelimab” as used herein, is an Fe engineered humanized monoclonal antibody (mAb) that binds to the human B-cell restricted surface antigen CD19 and has enhanced Fc binding to Fcγ receptor IIb (FcγRIIb). The molecule is an IgG1 immunoglobulin with a kappa light chain and 2 amino acid substitutions in the constant portion of the heavy chain. Obexelimab is a monoclonal antibody with a projected mass of approximately 147,426 Da based on the amino acid sequence. The heavy and light chains of obexelimab are given by SEQ ID NO: 10, and SEQ ID NO: 9, respectively.

Recurrence-free period: The term “recurrence-free period” as used herein, is defined as the time interval between the start of administration of an anti-CD19 antibody (e.g., obexelimab) to the time of recurrence of disease flare.

Rescue Therapy: The term “rescue therapy” as used herein refers to use of a therapy to treat a suspected remerging disease flare or worsening of disease symptoms. In some embodiments, the rescue therapy is a different approved therapy. In some embodiments, the rescue therapy is any therapy that may be used to lessen the symptoms associated with IgG4-RD disease flare. A common rescue therapy for IgG4-RD is glucocorticoid (GC) rescue therapy. Other rescue therapies include rituximab, and other anti-CD19 antibody therapies, to name but a few.

### Obexelimab and Variants Thereof

According to the present invention, Obexelimab and a variant thereof is used to treat a human patient suffering from an IgG4-RD. Obexelimab is a non-cytolytic CD-19 directed antibody for the treatment of active IgG4-RD (immunoglobulin G4 related diseases) and reduction in risk of flare recurrence in adult patients. In one aspect, the present invention provides a method of administering obexelimab for the reduction in risk of flare recurrence in adult patients. In one aspect, the present invention provides a method of administering obexelimab for the reduction in risk of flare recurrence in adult patients with IgG4-RD (immunoglobulin G4 related diseases). In one aspect, the present invention provides a method of administering obexelimab for the treatment of active IgG4-RD (immunoglobulin G4 related diseases) and reduction in risk of flare recurrence in adult patients.

Obexelimab is a monoclonal antibody specific for CD19 comprising: a light chain comprising a variable region having:

(SEQ ID NO: 2)
a CDR1 comprising RSSKSLQNVNGNTYLY,
(SEQ ID NO: 3)
a CDR2 comprising RMSNLNS,
and
(SEQ ID NO: 4)
a CDR3 comprising MQHLEYPIT;

a heavy chain comprising a variable region having

(SEQ ID NO: 5)
a CDR1 comprising SYVMH,
(SEQ ID NO: 6)
a CDR2 comprising WIGYINPYNDGTKY,
and
(SEQ ID NO: 7)
a CDR3 comprising GTYYYGTRVFDY,

wherein the heavy chain comprises amino acid substitutions in the Fc region S267E and L328F as compared to SEQ ID NO: 8:

(SEQ ID NO: 8)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK,

wherein the numbering is according to the EU index, as in Kabat.

In an embodiment, Obexelimab comprises: a light chain comprising an amino acid sequence of (SEQ ID NO: 9) and heavy chain comprising an amino acid sequence of, (SEQ ID NO: 10), as given by Table 1.

TABLE 1
Sequence of Heavy chain and Light chain amino acid sequence of
Obexelimab

| | |
|---|---|
| Heavy Chain | Light Chain |
| EVQLVESGGGLVKPGGSLKLSCAASGYTFT | DIVMTQSPATLSLSPGERATLSCRSSKSLQNVN |
| SYVMHWVRQAPGKGLEWIGYINPYNDGTKY | GNTYLYWFQQKPGQSPQLLIYRMSNLNSGVPDR |
| NEKFQGRVTISSDKSISTAYMELSSLRSED | FSGSGSGTEFTLTISSLEPEDFAVYYCMQHLEY |
| TAMYYCARGTYYYGTRVFDYWGQGTLVTVS | PITFGAGTKLEIKRTVAAPSVFIFPPSDEQLKS |
| SASTKGPSVFPLAPSSKSTSGGTAALGCLV | GTASVVCLLNNFYPREAKVQWKVDNALQSGNSQ |
| KDYFPEPVTVSWNSGALTSGVHTFPAVLQS | ESVTEQDSKDSTYSLSSTLTLSKADYEKHKVYA |
| SGLYSLSSVVTVPSSSLGTQTYICNVNHKP | CEVTHQGLSSPVTKSFNRGEC (SEQ ID NO: |
| SNTKVDKKVEPKSCDKTHTCPPCPAPELLG | 9) |
| GPSVFLFPPKPKDTLMISRTPEVTCVVVDV | |
| EHEDPEVKFNWYVDGVEVHNAKTKPREEQY | |
| NSTYRVVSVLTVLHQDWLNGKEYKCKVSNK | |
| AFPAPIEKTISKAKGQPREPQVYTLPPSRE | |
| EMTKNQVSLTCLVKGFYPSDIAVEWESNGQ | |
| PENNYKTTPPVLDSDGSFFLYSKLTVDKSR | |
| WQQGNVFSCSVMHEALHNHYTQKSLSLSPG | |
| K (SEQ ID NO: 10) | |

Variable region

| | |
|---|---|
| EVQLVESGGGLVKPGGSLKLSCAASGYTFT | DIVMTQSPATLSLSPGERATLSCRSSKSLQNVN |
| SYVMHWVRQAPGKGLEWIGYINPYNDGTKY | GNTYLYWFQQKPGOSPQLLIYRMSNLNSGVPDR |
| NEKFQGRVTISSDKSISTAYMELSSLRSED | FSGSGSGTEFTLTISSLEPEDFAVYYCMQHLEY |
| TAMYYCARGTYYYGTRVFDYWGQGTLVTVS | PITFGAGTKLEIK (SEQ ID NO: 11) |
| S (SEQ ID NO: 12) | |

In one embodiment, Obexelimab comprises a light chain variable region comprising SEQ ID NO: 11 and a heavy chain variable region comprising SEQ ID NO: 12.

Obexelimab works by exploiting the regulation of B-cell receptor (BCR) signaling by FcγRIIB1. Obexelimab binds CD19 of the BCR complex and its Fc is engineered to increase its affinity for the inhibitory FcγRIIB. Since CD19 is associated with the BCR, Obexelimab tethering of CD19 to FcγRIIB on the same cell poises the BCR complex for inhibition upon antigen-induced BCR aggregation. Obexelimab capitalizes upon the natural inhibitory mechanism of FcγRIIb, the only Fc receptor expressed by B cells, which acts as a negative regulator in conditions of antigen excess and immune complex formation (Chu et al., 2014). Obexelimab may also have an improved safety profile compared to B cell depleting antibodies as it may not mediate B cell killing.

### Variants

In an embodiment, a variant of Obexelimab is an immunoglobulin specific for CD19 comprises: a light chain comprising a variable region having a CDR1 comprising SEQ ID NO: 2, a CDR2 comprising SEQ ID NO: 3, and a CDR3 comprising SEQ ID NO: 4; and a heavy chain comprising a variable region having a CDR1 comprising SEQ ID NO: 5, a CDR2 comprising SEQ ID NO: 6, and a CDR3 comprising SEQ ID NO: 7, wherein the heavy chain comprises amino acid substitutions in the Fc region S267E and L328F as compared to SEQ ID NO: 8, wherein the numbering is according to the EU index, as in Kabat.

In some embodiments, a variant of obexelimab comprises a heavy chain variable region (VH) and/or a light chain variable region (VL), comprising a CDR1, a CDR2, and a CDR3, each of which differs by no more than 1, 2, 3, 4 or 5 amino acid residues from each of SEQ ID NO: 2, SEQ ID NO: 3, SEQ ID NO: 4, SEQ ID NO: 5, SEQ ID NO: 6 and/or SEQ ID NO: 7.

In an embodiment, the variant of obexelimab comprises: a light chain comprising an amino acid sequence 70%, 75%, 80%, 85%, 90%, 95%, 98% or 99% identical to of SEQ ID NO: 9 and/or amino acid substitutions in the Fc region S267E and L328F as compared to SEQ ID NO: 10, wherein the numbering is according the EU index, as in Kabat.

In some embodiments, a suitable variant of obexelimab binds to the same epitope on human CD19, as an antibody comprising a light chain of SEQ ID NO: 9 and a heavy chain of SEQ ID NO: 10. Epitope binding may be determined by a method known in the art.

In some embodiments, a suitable variant of obexelimab competes for binding to human CD19, as an antibody comprising a light chain of SEQ ID NO: 9 and a heavy chain of SEQ ID NO: 10, under a binning assay known in the art. As used herein, a binning assay refers to any method to regionally map the epitope to which the antibody binds. Standard methods for such antibody characterization, also known as epitope binning, typically involve surface plasmon resonance (SPR) technology. Using SPR, monoclonal antibody candidates are screened pairwise for binding to a target protein. Other standard methods involve ELISA-based screens and may require synthesis of sets of overlapping peptides corresponding to the protein of interest.

In some embodiments, the human CD19 comprises an amino acid sequence of SEQ ID NO: 1:

(SEQ ID NO: 1)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQOMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGODQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR

### Fc Receptor Binding Properties

Anti-CD19 antibodies (e.g., obexelimab) disclosed herein comprise an Fc variant that has enhanced Fc binding to the inhibitory Fcγ receptor IIb (FcγRIIb). Fe γ RIIb, the only FcR on B cells, serves as an antibody-sensing down-regulator of humoral immunity that is naturally engaged by immune complexes. When sufficient antibody is raised against a given antigen, specific immune complexes form and co-engage Fc γ RIIb and the B cell receptor (BCR) with high avidity, selectively suppressing only B cells recognizing cognate antigen. In addition, Fc γ RIIb regulates the activity of other B cell stimulators including interleukin (IL)-4, LPS, and BAFF that amplify BCR-driven proliferation and differentiation. By simultaneously binding CD19 and Fc γ RIIb, obexelimab (and variants described herein) mimics the action of antigen-antibody complexes and down-regulates B cell activity.

The Fc variants disclosed herein may be optimized for a variety of Fc receptor binding properties. An Fc variant that is engineered or predicted to display one or more optimized properties is herein referred to as an “optimized Fc variant.” Properties that may be optimized include but are not limited to enhanced or reduced affinity for an FcγR. In one embodiment, the Fc variants disclosed herein are optimized to possess enhanced affinity for an inhibitory receptor FcγRIIb. In other embodiments, immunoglobulins disclosed herein provide enhanced affinity for FcγRIIb, yet reduced affinity for one or more activating FcγRs, including for example FcγRI, FcγRIIa, FcγRIIIa, and/or FcγRIIIb. The FcγR receptors may be expressed on cells from any organism, including but not limited to human, cynomolgus monkeys, and mice. The Fc variants disclosed herein may be optimized to possess enhanced affinity for human FcγRIIb.

An Fc variant comprises one or more amino acid modifications relative to a parent Fc polypeptide, wherein the amino acid modification(s) provide one or more optimized properties. An Fc variant disclosed herein differs in amino acid sequence from its parent by virtue of at least one amino acid modification. Thus, Fc variants disclosed herein have at least one amino acid modification compared to the parent. Alternatively, the Fc variants disclosed herein may have more than one amino acid modification as compared to the parent, for example from about two to fifty amino acid modifications, e.g., from about two to ten amino acid modifications, from about two to about five amino acid modifications, etc. compared to the parent. Thus, the sequences of the Fc variants and those of the parent Fc polypeptide are substantially homologous. For example, the variant Fc variant sequences herein will possess about 80% homology with the parent Fc variant sequence, e.g., at least about 90% homology, at least about 95% homology, at least about 98% homology, at least about 99% homology, etc. Modifications disclosed herein include amino acid modifications, including insertions, deletions, and substitutions. Modifications disclosed herein also include glycoform modifications.

Modifications may be made genetically using molecular biology or may be made enzymatically or chemically.

Fc variants disclosed herein are defined according to the amino acid modifications that compose them. Thus, for example, S267E is an Fc variant with the substitution S267E relative to the parent Fc polypeptide. Likewise, S267E/L328F defines an Fc variant with the substitutions S267E and L328F relative to the parent Fc polypeptide. The identity of the WT amino acid may be unspecified, in which case the aforementioned variant is referred to as 267E/328F. It is noted that the order in which substitutions are provided is arbitrary, that is to say that, for example, 267E/328F is the same Fc variant as 328F/267E, and so on. Unless otherwise noted, positions discussed herein are numbered according to the EU index as described in Kabat (Kabat et al., 1991, Sequences of Proteins of Immunological Interest, 5th Ed., United States Public Health Service, National Institutes of Health, Bethesda, hereby entirely incorporated by reference). In brief, EU is the name of the first antibody molecule whose entire amino acid sequence was determined (Edelman et al., 1969, Proc Natl Acad Sci USA 63:78-85, hereby entirely incorporated by reference), and its amino acid sequence has become the standard numbering scheme for heavy chain constant regions. The EU protein has become the standard reference for defining numbering. Kabat et al. lists the EU sequence in a set of indices aligning it with other antibody sequences, serving as a necessary tool for aligning antibodies to the EU numbering scheme. Thus, as appreciated by those of skill in the art, the standard way of referencing the EU numbering is to refer to Kabat et al.'s alignment of sequences, because it puts EU in context with antibodies of other variable domain lengths. As such, as used herein, “the EU index as in Kabat” or “numbering is according to the EU index, as in Kabat” refers to the numbering of the EU antibody as described in Kabat.

In certain embodiments, the Fc variants disclosed herein are based on human IgG sequences, and thus human IgG sequences are used as the “base” sequences against which other sequences are compared, including but not limited to sequences from other organisms, for example rodent and primate sequences. Immunoglobulins may also comprise sequences from other immunoglobulin classes such as IgA, IgE, IgGD, IgGM, and the like. It is contemplated that, although the Fc variants disclosed herein are engineered in the context of one parent IgG, the variants may be engineered in or “transferred” to the context of another, second parent IgG. This is done by determining the “equivalent” or “corresponding” residues and substitutions between the first and second IgG, typically based on sequence or structural homology between the sequences of the first and second IgGs. In order to establish homology, the amino acid sequence of a first IgG outlined herein is directly compared to the sequence of a second IgG. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first immunoglobulin are defined. Alignment of conserved residues may conserve 100% of such residues. However, alignment of greater than 75% or as little as 50% of conserved residues is also adequate to define equivalent residues. Equivalent residues may also be defined by determining structural homology between a first and second IgG that is at the level of tertiary structure for IgGs whose structures have been determined. In this case, equivalent residues are defined as those for which the atomic coordinates of two or more of the main chain atoms of a particular amino acid residue of the parent or precursor (N on N, CA on CA, C on C and O on O) are within about 0.13 nm, after alignment. In another embodiment, equivalent residues are within about 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent IgG in which the IgGs are made, what is meant to be conveyed is that the Fc variants discovered as disclosed herein may be engineered into any second parent IgG that has significant sequence or structural homology with the Fc variant. Thus, for example, if a variant antibody is generated wherein the parent antibody is human IgG1, by using the methods described above or other methods for determining equivalent residues, the variant antibody may be engineered in another IgG1 parent antibody that binds a different antigen, a human IgG2 parent antibody, a human IgA parent antibody, a mouse IgG2a or IgG2b parent antibody, and the like. Again, as described above, the context of the parent Fc variant does not affect the ability to transfer the Fc variants disclosed herein to other parent IgGs.

The term “greater affinity” or “improved affinity” or “enhanced affinity” or “better affinity” than a parent Fc polypeptide, as used herein is meant that an Fc variant binds to an Fc receptor with a significantly higher equilibrium constant of association (KA or Ka) or lower equilibrium constant of dissociation (KD or Kd) than the parent Fc polypeptide when the amounts of variant and parent polypeptide in the binding assay are essentially the same. For example, the Fc variant with improved Fc receptor binding affinity may display from about 5 fold to about 1000 fold, e.g. from about 10 fold to about 500 fold improvement in Fc receptor binding affinity compared to the parent Fc polypeptide, where Fc receptor binding affinity is determined, for example, by the binding methods disclosed herein, including but not limited to Biacore, by one skilled in the art. Accordingly, by “reduced affinity” as compared to a parent Fc polypeptide as used herein is meant that an Fc variant binds an Fc receptor with significantly lower KA or higher KD than the parent Fc polypeptide. Greater or reduced affinity can also be defined relative to an absolute level of affinity. For example, according to the data herein, WT (native) IgG1 binds FcγRIIb with an affinity of about 1.5 mM, or about 1500 nM. Furthermore, some Fc variants described herein bind FcγRIIb with an affinity about 10-fold greater to WT IgG1. As disclosed herein, greater or enhanced affinity means having a KD lower than about 100 nM, for example between about 10 nM-about 100 nM, between about 1-about 100 nM, or less than about 1 nM.

In one embodiment, the Fc variants provide selectively enhanced affinity to FcγRIIb relative to one or more activating receptors. Selectively enhanced affinity means either that the Fc variant has improved affinity for FcγRIIb relative to the activating receptor(s) as compared to the parent Fc polypeptide but has reduced affinity for the activating receptor(s) as compared to the parent Fc polypeptide, or it means that the Fc variant has improved affinity for both FcγRIIb and activating receptor(s) as compared to the parent Fc polypeptide, however the improvement in affinity is greater for FcγRIIb than it is for the activating receptor(s). In alternate embodiments, the Fc variants reduce or ablate binding to one or more activating FcγRs, reduce or ablate binding to one or more complement proteins, reduce or ablate one or more FcγR-mediated effector functions, and/or reduce or ablate one or more complement-mediated effector functions.

The presence of different polymorphic forms of FcγRs provides yet another parameter that impacts the therapeutic utility of the Fc variants disclosed herein. Whereas the specificity and selectivity of a given Fc variant for the different classes of FcγRs significantly affects the capacity of an Fc variant to target a given antigen for treatment of a given disease, the specificity or selectivity of an Fc variant for different polymorphic forms of these receptors may in part determine which research or pre-clinical experiments may be appropriate for testing, and ultimately which patient populations may or may not respond to treatment. Thus the specificity or selectivity of Fc variants disclosed herein to Fc receptor polymorphisms, including but not limited to FcγRIIa, FcγRIIIa, and the like, may be used to guide the selection of valid research and pre-clinical experiments, clinical trial design, patient selection, dosing dependence, and/or other aspects concerning clinical trials.

Fc variants disclosed herein may comprise modifications that modulate interaction with Fc receptors other than FcγRs, including but not limited to complement proteins, FcRn, and Fc receptor homologs (FcRHs). FcRHs include but are not limited to FcRFH, FcRH2, FcRH3, FcRH4, FcRH5, and FcRH6 (Davis et al., 2002, Immunol. Reviews 190:123-136).

An important parameter that determines the most beneficial selectivity of a given Fc variant to treat a given disease is the context of the Fc variant. Thus the Fc receptor selectivity or specificity of a given Fc variant will provide different properties depending on whether it composes an antibody, Fc fusion, or Fc variants with a coupled fusion partner. In one embodiment, an Fc receptor specificity of the Fc variant disclosed herein will determine its therapeutic utility. The utility of a given Fc variant for therapeutic purposes will depend on the epitope or form of the target antigen and the disease or indication being treated. For some targets and indications, greater FcγRIIb affinity and reduced activating FcγR-mediated effector functions may be beneficial. For other target antigens and therapeutic applications, it may be beneficial to increase affinity for FcγRIIb, or increase affinity for both FcγRIIb and activating receptors.

### Formulation and Pharmaceutical Compositions

The present invention provides pharmaceutical compositions and formulations of anti-CD19 antibodies (e.g., obexelimab). Formulations of the anti-CD19 antibody disclosed herein are prepared for storage by mixing said antibody having the desired degree of purity with optional pharmaceutically acceptable carriers, excipients or stabilizers (Remington's Pharmaceutical Sciences 16th edition, Osol, A. Ed., 1980, incorporated entirely by reference), in the form of lyophilized formulations or aqueous solutions.

In some embodiments, pharmaceutical compositions of interest comprise an anti-CD19 antibody (e.g., obexelimab) at various concentrations. In some embodiments, suitable formulations may comprise the antibody of interest at a concentration up to about 250 mg/ml (e.g., up to about 225 mg/ml, up to 200 mg/ml, up to 150 mg/ml, up to 140 mg/ml, up to 130 mg/ml, up to 125 mg/ml, up to 120 mg/ml, up to 115 mg/ml, up to 110 mg/ml, up to 105 mg/ml, up to 100 mg/ml, up to 90 mg/ml, up to 80 mg/ml, up to 70 mg/ml, up to 60 mg/ml, up to 50 mg/ml, up to 40 mg/ml, up to 30 mg/ml, up to 25 mg/ml, up to 20 mg/ml, up to 10 mg/ml).

In some embodiments, suitable formulations may contain the anti-CD19 antibody at a concentration ranging between about 10-300 mg/ml (e.g., about 10-250 mg/ml, about 10-200 mg/ml, about 10-180 mg/ml, about 10-160 mg/ml, about 10-150 mg/ml, about 10-140 mg/ml, about 10-130 mg/ml, about 10-125 mg/ml, about 100-125 mg/ml, about 100-180 mg/ml, about 100-150 mg/ml, about 100-130 mg/ml, about 100-125 mg/ml, about 100-170 mg/ml, about 100-160 mg/ml, about 100-150 mg/ml, about 100-200 mg/ml, about 120-130 mg/ml).

In some embodiments, formulations suitable for subcutaneous administration may contain a protein of interest at a concentration of approximately 100 mg/ml, 115 mg/ml, 120 mg/ml, 125 mg/ml, 130 mg/ml, 135 mg/ml, 140 mg/ml, 145 mg/ml, 150 mg/ml, 200 mg/ml or 300 mg/ml.

In some embodiments, isotonic solutions are used. In some embodiments, slightly hypertonic solutions (e.g., up to 300 mM (e.g., up to 250 mM, 200 mM, 175 mM, 150 mM, 125 mM) sodium chloride in 5 mM sodium phosphate at pH 7.0) and sugar-containing solutions (e.g., up to 3% (e.g., up to 2.4%, 2.0%, 1.5%, 1.0%) sucrose in 5 mM sodium phosphate at pH 7.0). In some embodiments, a suitable formulation composition is saline (e.g., 150 mM NaCl in water).

Many therapeutic agents, and in particular the antibodies of the present invention, require controlled pH and specific excipients to maintain their solubility and stability in the pharmaceutical compositions of the present invention.

The pH of the pharmaceutical composition is an additional factor which is capable of altering the solubility of an anti-CD19 antibody (e.g., obexelimab) in an aqueous pharmaceutical composition. In some embodiments, pharmaceutical compositions of the present invention contain one or more buffers. In some embodiments, compositions according to the invention contain an amount of buffer sufficient to maintain the optimal pH of said composition between about 4.0-8.0, between about 5.0-7.5, between about 5.5-7.0, between about 6.0-7.0 and between about 6.0-7.5. In other embodiments, the buffer comprises up to about 50 mM (e.g., up to about 45 mM, 40 mM, 35 mM, 30 mM, 25 mM, 20 mM, 15 mM, 10 mM, 5 mM) of sodium phosphate. Suitable buffers include, for example acetate, succinate, citrate, phosphate, other organic acids and tris (hydroxymethyl) aminomethane (“Tris”).

Suitable buffer concentrations can be from about 1 mM to about 100 mM, or from about 3 mM to about 20 mM, depending, for example, on the buffer and the desired isotonicity of the formulation. In some embodiments, a suitable buffering agent is present at a concentration of approximately 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, formulations contain an isotonicity agent to keep the formulations isotonic. Exemplary isotonicity agents include, but are not limited to, glycine, sorbitol, mannitol, sodium chloride and arginine. In some embodiments, suitable isotonic agents may be present in formulations at a concentration from about 0.01-5% (e.g., 0.05, 0.1, 0.15, 0.2, 0.3, 0.4, 0.5, 0.75, 1.0, 1.25, 1.5, 2.0, 2.5, 3.0, 4.0 or 5.0%) by weight.

In some embodiments, formulations may contain a stabilizing agent to protect the antibody. Typically, a suitable stabilizing agent is a non-reducing sugar such as sucrose, raffinose, trehalose, or amino acids such as glycine, arginine and methionine. The amount of stabilizing agent in a formulation is generally such that the formulation will be isotonic. However, hypertonic formulations may also be suitable. In addition, the amount of stabilizing agent must not be too low such that an unacceptable amount of degradation/aggregation of the antibody occurs. Exemplary stabilizing agent concentrations in the formulation may range from about 1 mM to about 400 mM (e.g., from about 30 mM to about 300 mM, and from about 50 mM to about 100 mM), or alternatively, from 0.1% to 15% (e.g., from 1% to 10%, from 5% to 15%, from 5% to 10%) by weight. In some embodiments, the ratio of the mass amount of the stabilizing agent and the therapeutic agent is about 1:1. In other embodiments, the ratio of the mass amount of the stabilizing agent and the therapeutic agent can be about 0.1:1, 0.2:1, 0.25:1, 0.4:1, 0.5:1, 1:1, 2:1, 2.6:1, 3:1, 4:1, 5:1, 10:1, or 20:1. In some embodiments, suitable for lyophilization, the stabilizing agent is also a lyoprotectants.

The pharmaceutical compositions, formulations and related methods of the invention are useful for delivering anti-CD19 antibodies (e.g., subcutaneously) and for the treatment of the associated diseases. The pharmaceutical compositions of the present invention are particularly useful for delivering anti-CD19 antibodies (e.g., obexelimab) to patients suffering from IgG4-RD.

In some embodiments, it is desirable to add a surfactant to formulations. Exemplary surfactants include nonionic surfactants such as Polysorbates (e.g., Polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristarnidopropyl-, palmidopropyl-, or isostearamidopropyl-dimethylamine; sodium methyl cocoyl-, or disodium methyl ofeyl-taurate; and the MONAQUAT™ series (Mona Industries, Inc., Paterson, N.J.), polyethyl glycol, polypropyl glycol, and copolymers of ethylene and propylene glycol (e.g., Pluronics, PF68, etc). Typically, the amount of surfactant added is such that it reduces aggregation of the protein and minimizes the formation of particulates or effervescences. For example, a surfactant may be present in a formulation at a concentration from about 0.001-0.5% (e.g., about 0.005-0.05%, or 0.005-0.01%). In particular, a surfactant may be present in a formulation at a concentration of approximately 0.005%, 0.01%, 0.02%, 0.1%, 0.2%, 0.3%, 0.4%, or 0.5%, etc.

In some embodiments, suitable formulations may further include one or more bulking agents, in particular, for lyophilized formylations. A “bulking agent” is a compound which adds mass to the lyophilized mixture and contributes to the physical structure of the lyophilized cake. For example, a bulking agent may improve the appearance of lyophilized cake (e.g., essentially uniform lyophilized cake). Suitable bulking agents include, but are not limited to, sodium chloride, lactose, mannitol, glycine, sucrose, trehalose, hydroxyethyl starch. Exemplary concentrations of bulking agents are from about 1% to about 10% (e.g., 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, and 10.0%).

Formulations in accordance with the present invention can be assessed based on product quality analysis, reconstitution time (if lyophilized), quality of reconstitution (if lyophilized), high molecular weight, moisture, and glass transition temperature. Typically, protein quality and product analysis include product degradation rate analysis using methods including, but not limited to, size exclusion HPLC (SE-HPLC), cation exchange-HPLC (CEX-HPLC), X-ray diffraction (XRD), modulated differential scanning calorimetry (mDSC), reversed phase HPLC (RP-HPLC), multi-angle light scattering (MALS), fluorescence, ultraviolet absorption, nephelometry, capillary electrophoresis (CE), SDS-PAGE, and combinations thereof. In some embodiments, evaluation of product in accordance with the present invention may include a step of evaluating appearance (either liquid or cake appearance).

Generally, formulations (lyophilized or aqueous) can be stored for extended periods of time at room temperature. Storage temperature may typically range from 0° C. to 45° C. (e.g., 4° C., 20° C., 25° C., 45° C. etc.). Formulations may be stored for a period of months to a period of years. Storage time generally will be 24 months, 12 months, 6 months, 4.5 months, 3 months, 2 months or 1 month. Formulations can be stored directly in the container used for administration, eliminating transfer steps.

Formulations can be stored directly in the lyophilization container (if lyophilized), which may also function as the reconstitution vessel, eliminating transfer steps. Alternatively, lyophilized product formulations may be measured into smaller increments for storage. Storage should generally avoid circumstances that lead to degradation of the proteins, including but not limited to exposure to sunlight, UV radiation, other forms of electromagnetic radiation, excessive heat or cold, rapid thermal shock, and mechanical shock.

In some embodiments, formulations according to the present invention are in a liquid or aqueous form. In some embodiments, formulations of the present invention are lyophilized. Such lyophilized formulations may be reconstituted by adding one or more diluents thereto prior to administration to a patient. Suitable diluents include, but are not limited to, sterile water, bacteriostatic water for injection and sterile saline solution. Preferably, upon reconstitution, the antibody contained therein is stable, soluble and demonstrates tolerability upon administration to a patient.

The pharmaceutical compositions of the present invention are characterized by their tolerability. As used herein, the terms “tolerable” and “tolerability” refer to the ability of the pharmaceutical compositions of the present invention to not elicit an adverse reaction in the patient to whom such composition is administered, or alternatively not to elicit a serious adverse reaction in the patient to whom such composition is administered. In some embodiments, the pharmaceutical compositions of the present invention are well tolerated by the patient to whom such compositions is administered.

Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition that comprises the antibody disclosed herein may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Some embodiments include at least one of the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The formulations to be used for in vivo administration (e.g., subcutaneous administration) may be sterile. In some embodiments, the formulation is sterilized by filtration through sterile filtration membranes.

In some embodiments, the anti-CD19 antibodies (e.g., obexelimab) disclosed herein are formulated for subcutaneous (SC) administration. In some embodiments, the anti-CD19 antibody (e.g., obexelimab) formulation for SC administration comprises one or more buffers, one or more tonicity modifiers, one or more solvents, and one or more surfactants. Nonlimiting examples of buffers include phosphate, citrate, acetate, glutamate, carbonate, tartrate, triethanolamine (TRIS), glycylglycine, histidine, glycine, lysine, arginine, and other organic acids. More specifically, non-limiting examples of buffers include HEPES sodium, MES, potassium phosphate, potassium thiocyanate, sterilant, TAE, TBE, ammonium sulfate/HEPES, BuffAR, sodium acetate, sodium carbonate, sodium citrate, sodium dihydrogen phosphate, disodium hydrogen phosphate, and sodium phosphate. Additionally, the buffer may be various hydrate forms. For example, the buffer may be a monohydrate, a dihydrate, a trihydrate, a tetrahydate, a pentahydrate, a hexahydrate, a heptahydrate, an octahydrate, a nonahydrate, a decahydrate, an undecahydrate, and a dodecahydrate. Occasionally, a hydrate may be fractional such as a hemihydrate or a sequihydrate. Nonlimiting examples of tonicity modifies include sodium chloride, acetic acid, L-proline, dextrose, mannitol, potassium chloride, glycerin, and glycerol. Non-limiting example of solvents include water, propylene glycol, polyethylene glycols, ethanol, dimethyl sulfoxide, N-methyl-2-pyrrolidone, glycofurol, Solketal™, glycerol formal, acetone, tetrahydrofurfuryl alcohol, diglyme, dimethyl isosorbide, and ethyl lactate. Non-limiting examples of solvents include polysorbates (e.g. polysorbate-20, polysorbate-80), polyoxyethylene sorbitan monooleate (Tween 80), sorbitan monooleate polyoxyethylene sorbitan monolaurate (Tween 20), sorbitan trioleate (span 85), lecithin, and polyoxyethylene polyoxypropylene copolymers (Pluronics, Pluronic F-68).

The amounts of anti-CD19 antibody (e.g., obexelimab), buffer, tonicity modifier, solvent, and surfactants may vary. In some embodiments, the anti-CD19 antibody (e.g., obexelimab) is formulated at a concentration of 125 mg/mL obexelimab, 2.35 mg/mL sodium acetate trihydrate, 0.17 mg/mL acetic acid (at density 1.053 g/mL), 30 mg/mL L-proline, 0.1 mg/mL polysorbate 80, pH 5.5. In some embodiments, the anti-CD19 antibody (e.g., obexelimab) is formulated at a concentration of 80-200 mg/mL obexelimab, 1.5-3 mg/mL sodium acetate trihydrate, 0.1-0.2 mg/mL acetic acid (at density 1.053 g/mL), 10-50 mg/mL L-proline, 0.05-0.2 mg/mL polysorbate 80, pH 5.0-6.0. In some embodiments, the anti-CD19 antibody (e.g., obexelimab) is formulated at a concentration of 122-127 mg/mL obexelimab, 2.0-2.5 mg/mL sodium acetate trihydrate, 0.15-0.19 mg/mL acetic acid (at density 1.053 g/mL), 25-35 mg/mL L-proline, 0.05-0.15 mg/mL polysorbate 80, pH 5.0-6.0.

In certain embodiments, a subcutaneous (SC) formulation comprises the anti-CD19 antibody (e.g., obexelimab), one or more buffers, one or more tonicity modifiers, one or more solvents, and one or more surfactants. In some embodiments, the buffer can be a sodium acetate buffer. For example, the buffer can be sodium acetate trihydrate. In an embodiment, the tonicity modifier can be acetic acid, L-proline, and combinations thereof. In another embodiment, the solvent is water.

In some embodiments, the surfactant is a polysorbate. In some embodiments, the polysorbate is polysorbate-80. In some embodiments, a SC formulation comprises the anti-CD19 antibody (e.g., obexelimab), sodium acetate trihydrate, acetic acid and L-proline, water, and polysorbate-80.

In some embodiments, the subcutaneous (SC) formulation comprises the anti-CD19 antibody (e.g., obexelimab) in an amount from about 1 mg to about 500 mg per mL or about 50 mg to about 250 mg per mL or about 100 mg to about 250 mg per mL, sodium acetate trihydrate in an amount from about 1 to about 10 mg per mL or about 1 to about 5 mg per mL or about 1 to about 2.5 mg per mL, acetic acid and L-proline in an amount from about 5 to about 50 mg per mL or about 10 to about 50 mg per mL or about 20 to about 40 mg per mL, water up to about 1 mL, and polysorbate-80 in an amount from about 0.01 mg to about 1 mg per mL or about 0.01 to about 0.5 mg/ml or about 0.05 to about 0.2 mg/ml. Specifically, a SC formulation comprises the anti-CD19 antibody (e.g., obexelimab) in an amount from about 100 mg to about 250 mg/ml, sodium acetate trihydrate in an amount from about 1 to about 2.5 mg/ml, acetic acid and L-proline in an amount from about 20 to about 40 mg/ml, water up to about 1 mg/ml, and polysorbate-80 in an amount from about 0.05 to about 0.2 mg per ml

The anti-CD19 antibodies (e.g., obexelimab) disclosed herein may also be formulated as immunoliposomes. A liposome is a small vesicle comprising various types of lipids, phospholipids and/or surfactant that is useful for delivery of an anti-CD19 antibody (e.g., obexelimab) to a mammal. Liposomes containing the anti-CD19 antibody (e.g., obexelimab) are prepared by methods known in the art. Liposomes with enhanced circulation time are disclosed in U.S. Pat. No. 5,013,556, incorporated entirely by reference. In some embodiments, the anti-CD19 antibody (e.g., obexelimab) is formulated in liposomes generated by the reverse phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

In some embodiments, the anti-CD19 antibody (e.g., obexelimab) is entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980, incorporated entirely by reference.

In some embodiments, the anti-CD19 antibody (e.g., obexelimab) sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides (U.S. Pat. No. 3,773,919, incorporated entirely by reference), copolymers of L-glutamic acid and gamma ethyl-L-glutamate, non-degradable ethylene-vinyl acetate, degradable lactic acid-glycolic acid copolymers such as the Lupron Depot® (which are injectable microspheres composed of lactic acid-glycolic acid copolymer and leuprolide acetate), poly-D-(−)-3-hydroxybutyric acid, and ProLease® (commercially available from Alkermes), which is a microsphere-based delivery system composed of the desired bioactive molecule incorporated into a matrix of poly-DL-lactide-co-glycolide (PLG).

### Containers for Injection

The present invention provides containers for injecting the pharmaceutical compositions and formulations of anti-CD19 antibodies (e.g., obexelimab). In one aspect, the container comprises a liquid pharmaceutical composition comprising obexelimab. Suitable containers include, without limitation, a syringe, an autoinjector, vial, infusion bottle, ampoule, carpoule, a syringe equipped with a needle protection system, and a carpoule within an injection pen.

In some embodiments, the container comprising the liquid pharmaceutical composition is a prefilled syringe, a vial, or an autoinjector. In some embodiments, the container is a prefilled syringe. In some embodiments the container is an autoinjector.

In some embodiments, the container is a prefilled syringe or autoinjector comprising a liquid pharmaceutical composition comprising:
a. 122-127 mg/mL obexelimab,
b. 2.0-2.5 mg/mL sodium acetate trihydrate, at a pH 5.0-6.0.
c. 0.15-0.19 mg/mL acetic acid (at density 1.053 g/mL),
d. 25-35 mg/mL L-proline,
e. 0.05-0.15 mg/mL polysorbate 80.

In some embodiments, the prefilled syringe or autoinjector comprises a liquid pharmaceutical composition comprising: the liquid pharmaceutical composition comprises about 125 mg/mL obexelimab, 2.35 mg/mL sodium acetate trihydrate, 0.17 mg/mL acetic acid, 30 mg/mL L-proline, 0.1 mg/mL polysorbate 80 at pH 5.5.

In some embodiments, the prefilled syringe or autoinjector facilitates subcutaneous or intradermal delivery of the pharmaceutical composition. In some embodiments, the method of treating IgG4-RD described herein comprises, administering the formulation comprising obexelimab into the patient's bloodstream following a single or multiple subcutaneous injection to the abdomen of the patient using the prefilled syringe or autoinjector.

In some embodiments, the composition in the prefilled syringe is stable for at least 3 months when stored at 2-8° C. In some embodiments, the composition in the pre-filled syringe is stable for at least 6 months when stored at 2-8° C. In some embodiments, the composition in the pre-filled syringe is stable for at least 6 months when stored at 2-8° C. In some embodiments, the composition in the pre-filled syringe is stable for at least 1 year when stored at 2-8° C. In some embodiments, the composition in the pre-filled syringe is stable for at least 2 years when stored at 2-8° C.

### IgG4-Related Disease (IgG4-RD)

IgG4-RD is a serious, rare, chronic fibro-inflammatory condition that typically affects multiple organs, leading to significant organ dysfunction and failure, and even death if the condition is not recognized and treated in a timely manner. In some embodiments, the disease commonly affects major organs such as the pancreas, liver, kidneys, lungs, and eyes. In some embodiments, patients may present with a single organ involved but more frequently present with multiple organ involvement. As the disease progresses and patients experience new or worsening signs/symptoms (flares), additional organs develop lesions, and the cellular inflammation characterizing early disease moves towards a more fibrotic stage, causing major tissue damage, and ultimately organ failure. For example, cholangitis due to IgG4-RD can lead to hepatic failure, IgG4-related (type 1) autoimmune pancreatitis can lead to failure of either or both the endocrine and exocrine pancreas, and IgG4-related aortitis can lead to aneurysms and/or aortic dissection.

Several B cell subsets have been described by flow cytometry to be elevated in peripheral blood of IgG4-RD patients (Wallace 2015, Mattoo 2014) and CD19+ plasmablasts have been shown to actively contribute to tissue fibrosis (Della-Torre 2019). Histopathologic features of IgG4-RD include lymphoplasmacytic infiltrate, storiform fibrosis and obliterative phlebitis (Carruthers 2015). The hallmark of IgG4-RD is IgG4-positive plasma cell infiltrations in the affected organs (Cheuk 2010, Floreani 2020).

Clinical symptoms and the course of disease are dependent on the affected organ(s) and may therefore be evaluated and treated by many different specialties including rheumatologists, gastroenterologists, nephrologists, and ophthalmologists. Despite the growing recognition of this disease and crucial advances in the understanding of its pathophysiology, there remains no approved medication. Indeed, no randomized, double-blind, placebo-controlled trial has ever been published in IgG4-RD. Treatment is based on the consensus of expert opinion (Khosroshahi 2015), yet treatment options remain few and are often limited by common co-morbidities among patients with IgG4-RD (e.g., elderly age, diabetes, obesity, hypertension) and the fact that the disease frequently targets organs such as the pancreas, exacerbating treatment complications associated with glucocorticoids.

B cell, including plasmablast-directed therapy with a CD19 monoclonal antibody, such as, Obexelimab (XmAb5871) which coengages CD19 and FcγRIIb, mimics the natural action of antigen-antibody complexes and down-regulates B cell activity. The proposed mechanism of action of obexelimab of simultaneously binding CD19 and FcγRIIb and down regulating B cell activity has been demonstrated in vitro and in vivo, including in classical animal models of autoimmune disease.

IgG4-RD is currently incurable. The goals of treatment are to reduce inflammation and swelling in the organs, prevent or reverse (if possible) fibrosis and increase glandular secretion. Immediate treatment is warranted to prevent organ failure when vital organs are involved. For example, cholangitis due to IgG4-RD can lead to hepatic failure, IgG4-related (type 1) autoimmune pancreatitis can lead to failure of either or both the endocrine and exocrine pancreas, and IgG4-related aortitis can lead to aneurysms and/or aortic dissection. At the present time, glucocorticosteroids are the first line of therapy. Although this approach is effective initially in most patients, the relapse rate upon tapering or discontinuation is high (Khosroshahi et al. 2015). In addition, the long-term use of glucocorticosteroids causes toxicity in patients, especially in older populations including osteoporosis, high blood pressure and diabetes.

The pathogenesis of IgG4-RD suggests that B cell targeted therapies may be useful therapeutic disease interventions. For example, results from a small pilot study of the anti-CD20 monoclonal antibody rituximab in 30 patients with IgG4-RD support this concept (Carruthers 2015). Rituximab and other B cell depleting agents can cause prolonged depletion of B cells for 6 months or longer, which places patients at risk of opportunistic infections and potentially reduces response to vaccines. Therefore, there is a need for new therapies that do not deplete B cells, but rather down-regulate their activity.

The IgG4-related diseases for treatment in the present disclosure can be selected from the group consisting of IgG4-related sialadenitis (chronic sclerosing sialadenitis, Kuttner's tumour, Mikulicz's disease), IgG4-related dacryoadenitis (Mikulicz's disease), IgG4-related ophthalmic disease (idiopathic orbital inflammatory disease, orbital pseudotumor), chronic sinusitis, eosinophilic angiocentric fibrosis, IgG4-related hypophysitis (IgG4-related panhypophysitis, IgG4-related adenohypophysitis, gG4-related infundibuloneurohypophysitis, autoimmune hypophysitis), IgG4-related pachymeningitis, IgG4-related leptomeningitis (idiopathic hypertrophic pachymeningitis), IgG4-related pancreatitis (Type 1 autoimmune pancreatitis, IgG4-related AIP, lymphoplasmacytic sclerosing pancreatitis, chronic pancreatitis with diffuse irregular narrowing of the main pancreatic duct), IgG4-related lung disease (Pulmonary inflammatory pseudotumour), IgG4-related pleuritis, IgG4-related hepatopathy, IgG4-related sclerosing cholangitis, IgG4-related cholecystitis, IgG4-related aortitis (inflammatory aortic aneurysm), IgG4-related periaortitis (chronic periaortitis), IgG4-related periarteritis, IgG4-related pericarditis, IgG4-related mediastinitis (fibrosing mediastinitis), IgG4-related retroperitoneal fibrosis (retroperitoneal fibrosis, Albarran-Ormond syndrome, Ormond's disease (tetroperitoneal fibrosis)), perirenal fasciitis, Gerota's fasciitis/syndrome, periureteritis fibrosa, sclerosing lipogranuloma, sclerosing retroperitoneal granuloma, non-specific retroperitoneal inflammation, sclerosing retroperitonitis, retroperitoneal vasculitis with perivascular fibrosis), IgG4-related mesenteritis (subtypes are: mesenteric panniculitis, mesenteric lipodystrophy and retractile mesenteritis) (sclerosing mesenteritis, systemic nodular panniculitis, liposclerosis mesenteritis, mesenteric Weber-Christian disease, mesenteric lipogranuloma, xanthogranulomatous mesenteritis), IgG4-related mastitis (sclerosing mastitis), IgG4-related kidney disease (IgG4-RKD), IgG4-related tubulointerstitial nephritis (IgG4-TIN), IgG4-related membranous glomerulonephritis (idiopathic tubulointerstitial nephritis), IgG4-related prostatitis, IgG4-related perivasal fibrosis (chronic orchialgia), IgG4-related paratesticular pseudotumor, IgG4-related epididymo-orchitis (paratesticular fibrous pseudotumor, inflammatory pseudotumor of the spermatic cord, pseudosarcomatous myofibroblastic proliferations of the spermatic cord, proliferative funiculitis, chronic proliferative periorchitis, fibromatous periorchitis, nodular periorchitis, reactive periorchitis, fibrous mesothelioma), IgG4-related lymphadenopathy, IgG4-related skin disease (angiolymphoid hyperplasia with eosinophilia, cutaneous pseudolymphoma), IgG4-related perineural disease, and IgG4-related thyroid disease (Reidel's thyroiditis), eosinophilic angiocentric fibrosis (affecting the orbits and upper respiratory tract), inflammatory pseudotumour, and multifocal fibrosclerosis.

Additionally, in some embodiments, the present disclosure also includes methods of treating manifestations of an IgG4-RD selected from the group consisting of autoimmune pancreatitis (lymphoplasmacytic scleorising pancreatitis), eosinophilic angiocentric fibrosis (affecting the orbits and upper respiratory tract), fibrosing mediastinitis, idiopathic hypertrophic pachymeningitis, idiopathic tubulointerstitial nephritis, inflammatory pseudotumour, Kuttner's tumour, Mikulicz's disease, fibrosclerosis, periaortitis, periarteritis, inflammatory aortic multifocal aneurysm, Ormond's disease (tetroperitoneal fibrosis), Riedel's thyroiditis, and sclerosing mesenteritis.

### Human Patient Population

Eligible human patients selected for receiving obexelimab, according to the invention, are 18 years of age or older that have active IgG4-RD. In some embodiments, eligible patients have active IgG4-RD signs/symptoms (e.g., flare) that require, the initiation of therapy. In some embodiments, the patient population is adults (≥18 years of age) with active IgG4-RD. In some embodiments, the patient is first assessed for the following factors:
1 organ involvement vs >1 organ involvement
Newly diagnosed vs recurring disease has met the classification criteria for participation
in clinical trials in IgG4-RD.

In some embodiments, patients have recent active disease requiring GC therapy. In some embodiments, treatment efficacy of obexelimab is determined by reducing the risk of flares compared to placebo control. Eligible patients may have ≥1 organ involvement. In some embodiments, the affected organ in IgG4-RD is lung. In some embodiments, the affected organ in IgG4-RD is aorta. In some embodiments, an affected organ in IgG4-RD is a retroperitoneum. In some embodiments, an affected organ in IgG4-RD is a pachymeninges. In some embodiments, an affected organ in IgG4-RD is a thyroid. In some embodiments, an affected organ in IgG4-RD is at least one orbit. In some embodiments, an affected organ in IgG4-RD is a lachrymal gland. In some embodiments, a organ in IgG4-RD is a salivary gland. In some embodiments, an affected organ in IgG4-RD is a liver. In some embodiments, an affected organ in IgG4-RD is a bile duct. In some embodiments, an affected organ in IgG4-RD is pancreas. In some embodiments, an affected organ in IgG4-RD is a kidney. In some embodiments, an affected organ in IgG4-RD is skin. In some embodiments, an affected organ in IgG4-RD is a thyroid. In some embodiments, an affected organ in IgG4-RD is a pituitary gland. In some embodiments, an affected organ in IgG4-RD is a lymph node. Patients with disease in only 1 organ system whose primary manifestation is fibrosis (i.e., retroperitoneum fibrosis without aortitis, Riedel's thyroiditis, fibrosing mediastinitis, sclerosing mesenteritis involvement, etc.) may be excluded. In some embodiments, organ involvement refers to enlargement or tumor-like mass in an affected organ except in (1) the bile ducts, where narrowing tends to occur, (2) the aorta, where wall thickening or aneurysmal dilatation is typical and (3) the lungs, which involve thickening of the bronchovascular bundles.

In some embodiments, the prior therapy is GC therapy and/or an increase in background long-term GC therapy.

In some embodiments, eligible patients have received at least 2 weeks of GC treatment. In some embodiments, eligible patients according to the invention, are required to receive at least 3 weeks of GC treatment. In some embodiments, eligible patients according to the invention, are required to receive more than 3 weeks of GC treatment. In some embodiments, eligible patients according to the invention, are required to receive up to a maximum of 6 weeks of GC treatment. In some embodiments, the GC treatment is administered at a dose of 30-50 mg/day prednisone or equivalent. In some embodiments, the GC treatment is administered at a dose of 10-70 mg/day prednisone or equivalent. In some embodiments, the GC treatment is administered at a dose of 20-60 mg/day prednisone or equivalent. Table 3 shows the equivalent doses of exemplary GCs.

In some embodiments, in eligible patients according to the invention, the GC therapy is administered along with obexelimab. In some embodiments, the GC treatment is tapered before the start of obexelimab therapy. Exemplary taper protocols involve administration of corticosteroids at 0.6-1.0 mg/kg daily for 2-4 weeks followed by a gradual taper (Khosroshahi 2015). In some exemplary embodiments, GC is completely tapered within 8-12 weeks, to discontinuation. In some embodiments the tapering takes less than 8 weeks. In some embodiments, the tapering takes more than 12 weeks. In some exemplary embodiments, GC treatment is allowed to continue at a low to moderate dose of 2.5-10.0 mg daily chronically (Kamisawa 2017).

In some embodiments, eligible patients according to the invention, can have plasmablast levels greater than 100 cells/mL, greater than 200 cells/mL, greater than 300 cells/mL, greater than 400 cells/mL, greater than 500 cells/mL, greater than 600 cells/mL, greater than 700 cells/mL, greater than 800 cells/mL, greater than 900 cells/mL, greater than 1000 cells/mL, greater than 2000 cells/mL, greater than 3000 cells/mL, greater than 4000 cells/mL or greater than 5000 cells/mL.

In some embodiments, eligible patients according to the invention, can have 2019 American College of Rheumatology (ACR)/European League Against Rheumatism (EULAR) Classification Criteria for IgG4-RD with a score of ≥20. The ACR/EULAR scoring system was developed by an international multispecialty group of 86 physicians assembled by the ACR and the EULAR. The scoring system uses consensus exercises, existing literature, derivation, and validation cohorts of 1,879 patients (1,086 cases, 793 mimickers), and multi-criterion decision analysis to identify, weight, and test potential classification criteria.

In some embodiments, eligible patients according to the invention have a recent active flare that requires treatment. In some embodiments, patients experiencing flare receive corticosteroids, followed by a protocol-specified taper on day of randomization.

In some embodiments, eligible patients according to the invention may have at least 1 organ involvement. In some embodiments, patients according to the invention may have more than 1 organ involvement. In some embodiments, patients with disease in only 1 organ system whose primary manifestation is fibrosis (i.e., retroperitoneum fibrosis without aortitis, Riedel's thyroiditis, fibrosing mediastinitis, sclerosing mesenteritis involvement, etc.) will be excluded.

Treatment of IgG4-RD with Obexelimab

There are no approved treatments for IgG4-RD and the goal of treatment has been to prevent inflammation, which can result in fibrosis and associated organ failure. Obexelimab (XmAb5871) is a humanized anti-CD19 monoclonal antibody with an Fc portion engineered for increased affinity to FcγRIIb, the only receptor on B-cells.

In some embodiments, administration of obexelimab in a subcutaneous (SC) formulation minimizes the risk of acute administration-related reactions.

In some embodiments, obexelimab is administered at 250 mg per week subcutaneously, at a concentration of 125 mg/ml, as 2×1 ml injections. In some embodiments, obexelimab is administered at 250 mg per week subcutaneously, at a concentration of 125 mg/ml, as 1×2 ml injection. In some embodiments, obexelimab is administered at 200 mg per week subcutaneously. In some embodiments, obexelimab is administered at 150 mg per week subcutaneously. In some embodiments, obexelimab is administered at 100 mg per week subcutaneously. In some embodiments, obexelimab is administered as a single injection. In some embodiments, obexelimab is administered as more than 2 injections, for example, obexelimab is administered as 3 injections, or 4 injections or more. In some embodiments, the concentration of obexelimab is 100 mg/ml. In some embodiments, the concentration of obexelimab is 50 mg/ml. In some embodiments, obexelimab is administered at a concentration of 100 mg/ml, as 2×1 ml injections, subcutaneously.

In one aspect, the present invention provides a method of treating IgG4-related disease (IgG4-RD), comprising administering obexelimab subcutaneously to a human patient ≥18 years of age at a dose of 250 mg per week.

In some embodiments, the method comprises administering obexelimab subcutaneously at a dose of 125 mg twice a week. In some embodiments, the method comprises administering obexelimab subcutaneously at a dose of 125 mg twice a week. In some embodiments, the method comprises administering obexelimab subcutaneously at a dose of 125 mg every 3 days. In some embodiments, the method comprises administering obexelimab subcutaneously at a dose of 250 mg every 7 days.

In some embodiments, obexelimab can be administered subcutaneously to a human patient >18 years of age at a dose of 200 mg once a week. In some embodiments, obexelimab can be administered subcutaneously to a human patient >18 years of age at a dose of 125 mg twice a week. In some embodiments, obexelimab is administered subcutaneously to a human patient >18 years of age at a dose of 100 mg twice a week. In some embodiments, obexelimab is administered subcutaneously to a human patient >18 years of age at a dose of 300 mg once a week. In some embodiments, obexelimab is administered subcutaneously to a human patient >18 years of age at a dose of 150 mg twice a week.

In some embodiments, obexelimab is administered in a liquid formulation comprising 125 mg/mL obexelimab. In some embodiments, obexelimab is administered as 2×1 mL injections. In some embodiments, obexelimab is administered as 2×1 mL injections for a total dose of 250 mg. In some embodiments, obexelimab injections are administered concurrently. In some embodiments, obexelimab injections are administered within 1 minute, 2 minutes, 3 minutes, 4 minutes, 5 minutes, 6 minutes, 7 minutes, 8 minutes, 9 minutes, 10 minutes, or within 20 minutes of each other. In some embodiments, obexelimab injections are administered within 1 hour, within 2 hours, within 3 hours, within 4 hours, within 5 hours, within 6 hours, within 7 hours, within 8 hours, within 9 hours, within 10 hours, within 11 hours, or within 12 hours of each other.

In some embodiments, obexelimab is administered in a liquid formulation comprising 125 mg/mL obexelimab as 4×0.25 mL injections. In some embodiments, obexelimab is administered in a liquid formulation comprising 125 mg/mL obexelimab as 4×0.25 mL injections for a total dose of 250 mg. In some embodiments, obexelimab injections are administered concurrently. In some embodiments, obexelimab injections are administered within 1 minute, 2 minutes, 3 minutes, 4 minutes, 5 minutes, 6 minutes, 7 minutes, 8 minutes, 9 minutes, 10 minutes, or within 20 minutes of each other. In some embodiments, obexelimab injections are administered within 1 hour, within 2 hours, within 3 hours, within 4 hours, within 5 hours, within 6 hours, within 7 hours, within 8 hours, within 9 hours, within 10 hours, within 11 hours, or within 12 hours of each other.

In some embodiments, obexelimab is administered in a liquid formulation as a single injection. In some embodiments, obexelimab is administered in a liquid formulation as a single injection for a total dose of 250 mg.

In some embodiments, obexelimab is administered at a dose of about 100-500 mg per week. In some embodiments, obexelimab is administered at a dose of about 100-200 mg per week, 100-300 mg per week, 100-400 mg per week, 200-400 mg per week, 100-300 mg per week, 200-350 mg per week, 150-300 mg per week, 125-275 mg per week, 225-275 mg per week, 230-260 mg per week, 240-260 mg per week, or 245-255 mg per week. In some embodiments, obexelimab is administered at a dose of about 100 mg per week, 150 mg per week, 200 mg per week, 250 mg per week, 300 mg per week, 350 mg per week, 400 mg per week, 450 mg per week, or 500 mg per week.

In some embodiments, the weekly dose may be injected at once or divided into two or more injections throughout a week. In some embodiments, the weekly dose is divided into 2 injections, 3 injections, 4 injections, 5 injections, 6 injections or daily injections. In some embodiments, the weekly dose is divided equally. In some embodiments, the weekly dose is divided unequally. In some embodiments, the weekly dose is divided such that it is administered every other day, every 2 days or every 3 days.

In some embodiments, a suitable dose ranges from 20-40 mg daily, 25-40 mg daily, or 30-40 mg daily. In some embodiments, a suitable dose is 35 mg daily.

In some embodiments, obexelimab is administered at a dose of 500 mg every two weeks. In some embodiments, obexelimab is administered every 2 weeks and the dose may be injected at once or divided into two or more injections. In some embodiments, the dose is divided into 2 injections, 3 injections, 4 injections, 5 injections, 6 injections or more injections.

In some embodiments, the patient receives 1000 mg of obexelimab per month. In some embodiments, the 1000 mg monthly dose may be injected at once or divided into 4 or more injections. In some embodiments, the dose is divided into 4 injections, 5 injections, 6 injections, 7 injections, 8 injections or more injections.

### IgG4-RD Flare

In some embodiments, IgG4-RD Flare is determined using IgG4-RD Flare Criteria. In some embodiments, IgG4-RD Flare Criteria is an objective, organ-specific instrument that details assessments and findings required to support an organ-specific flare diagnosis, based on patient-reported symptoms, physical examinations, biopsy, imaging, and laboratory findings. In some embodiments, the IgG4-RD flare criteria minimizes subjectivity in identification of disease flares by investigators. In some embodiments, IgG4-RD flare is evaluated using methods known in the art. In some embodiments, flare is diagnosed by analyzing serum IgG4 values, which may be normal in some patients with IgG4-RD. In some embodiments, IgG4-RD Flare Criteria includes collection of data for flare evaluation described below.

In some embodiments, the recurrence-free period will be evaluated compared to placebo or compared to before start of the treatment. In some embodiments, increase in the recurrence-free period is considered a positive outcome. As explained above, the recurrence-free period may be determined by the days to development of a disease flare.

### Time to Disease Flare (TDF)

In some embodiments, time to disease flare is a primary end point. In some embodiments, time to first IgG4-RD flare that requires initiation of rescue therapy. In some embodiments, during the clinical trial, TDF is the recurrence-free time from the start of the treatment to the reappearance of previous signs/symptoms or appearance of new signs/symptoms of IgG4-RD. For example, if a subject has recurrence of IgG4-RD flare 3 weeks after commencement of the obexelimab treatment, the TDF for the subject is 3 weeks. In some embodiments, from the TDF is measured up to week 52. In some embodiments, TDF is an assessment to evaluate the effect of weekly SC administration of obexelimab in reducing the risk of flares in patients with active IgG4-RD. In some embodiments, TDF is significantly longer in patients treated with obexelimab compared to patients who received placebo.

In some embodiments recurrence-free period is one week. In some embodiments recurrence-free period is one week. In some embodiments recurrence-free period is two weeks. In some embodiments recurrence-free period is three weeks. In some embodiments recurrence-free period is four weeks. In some embodiments recurrence-free period is one week. In some embodiments recurrence-free period is one month. In some embodiments recurrence-free period is one week. In some embodiments recurrence-free period is two months. In some embodiments recurrence-free period is three months. In some embodiments recurrence-free period is four months. In some embodiments recurrence-free period is five months. In some embodiments recurrence-free period is six months. In some embodiments recurrence-free period is seven months. In some embodiments recurrence-free period is nine months. In some embodiments recurrence-free period is ten months. In some embodiments recurrence-free period is eleven months. In some embodiments recurrence-free period is one year or more.

### Collection of Data for Flare Evaluation (Flare Criteria)

In some embodiments, obexelimab administration results in reduction in the reoccurrence of flare. Although flares may be treated with GCs, steroids do not prevent recurrence of disease after their discontinuation (Kamisawa 2017, Raina 2009). In some embodiments, the IgG4-RD flare is assessed at clinical visits. In some embodiments, if a patient demonstrates reappearance of prior sign/symptoms or new signs/symptoms of IgG4-RD, a physical examination, imaging, and/or biochemical parameters specific to the area of flare should be obtained. A comprehensive account of the assessments and information is provided by the investigator, in addition to testing serum IgG4 levels. Exemplary assessment data include but are not limited to:
Earliest date of onset of flare signs/symptoms
Organ specific evaluation of flare:
   Patient reported symptoms
   Physical examination findings
   Laboratory findings
   Imaging findings
   Biopsy findings
   Incidental finding during surgery
Investigator decision on whether flare that requires rescue therapy has occurred
Treatment
   Treatment initiated (Y/N)
   Treatment initiation date
   Treatment type
Glucocorticoid steroids
Immunosuppressant other than steroids
Surgical intervention, including stenting
   Duration of treatment
Outcome of treatment compared to pre-flare baseline
   Resolved without sequelae
   Resolved with sequalae
   Improved but still present
   Unchanged
   Worsened since treatment
Narrative of entire event including description of current signs and symptoms and any relevant historical data.

### Rescue Therapy

In the event of a disease flare, patients may be administered a rescue therapy for acute treatment of the flare. The rescue therapy involves administration of GC therapy. In some embodiments, rescue therapy involved administration of <40 mg/day prednisone or equivalent for <8 weeks in combination with obexelimab at a dose of 250 mg every 7 days.

In some embodiments, the GC therapy is administered at a dose of 20-60 mg/day prednisone or equivalent. In some embodiments, the GC therapy is administered at a dose of 10-100 mg/day prednisone or equivalent. In some embodiments, the GC therapy is administered at a dose of about 1-70 mg/day, about 5-70 mg/day, about 10-70 mg/day, about 15-70 mg/day, about 20-70 mg/day, about 25-70 mg/day, about 30-70 mg/day, about 35-70 mg/day, about 40-70 mg/day prednisone or equivalent. In some embodiments, the GC therapy is administered at a dose of about 1-60 mg/day, about 5-60 mg/day, about 10-60 mg/day, about 15-60 mg/day, about 20-60 mg/day, about 25-60 mg/day, about 30-60 mg/day, about 35-60 mg/day, about 40-60 mg/day prednisone or equivalent.

In some embodiments, the GC therapy is administered at a dose of about 1-150 mg/day, about 5-150 mg/day, about 10-150 mg/day, about 15-150 mg/day, about 20-150 mg/day, about 25-150 mg/day, about 30-150 mg/day, about 35-150 mg/day, about 40-150 mg/day, about 45-150 mg/day, about 50-150 mg/day, about 55-150 mg/day, about 60-150 mg/day, about 65-150 mg/day, about 70-150 mg/day, about 75-150 mg/day, about 80-150 mg/day, about 90-150 mg/day, or about 100-150 mg/day, prednisone or equivalent. In some embodiments, the GC therapy is administered at a dose of about 5-120 mg/day, about 5-110 mg/day, about 10-90 mg/day, about 15-100 mg/day, about 20-100 mg/day, about 25-100 mg/day, about 30-100 mg/day, about 35-100 mg/day, about 40-100 mg/day, about 45-100 mg/day, about 50-100 mg/day, about 55-100 mg/day, about 60-100 mg/day, about 65-100 mg/day, about 70-100 mg/day, about 75-100 mg/day, about 80-100 mg/day, or about 90-100 mg/day prednisone or equivalent.

In some embodiments, the GC therapy is administered at a dose of up to about 150 mg/day, up to about 120 mg/day, up to about 110 mg/day, up to about 100 mg/day, up to about 90 mg/day, up to about 80 mg/day, up to about 70 mg/day, up to about 60 mg/day, up to about 50 mg/day, up to about 40 mg/day, up to about 30 mg/day, up to about 20 mg/day, up to about 15 mg/day, up to about 10 mg/day, up to about 5 mg/day, or up to about 1 mg/day prednisone or equivalent.

In some embodiments, the GC therapy is administered at a dose of 0.1-1 mg/kg/day, a dose of 0.1-0.8 mg/kg/day, a dose of 0.1-0.7 mg/kg/day, a dose of 0.1-0.6 mg/kg/day, a dose of 0.1-0.5 mg/kg/day, a dose of 0.1-0.4 mg/kg/day, a dose of 0.1-0.3 mg/kg/day, a dose of 0.1-0.2 mg/kg/day or a dose of 0.05-0.1 mg/kg/day prednisone or equivalent.

In some embodiments, the GC therapy is administered at a dose of up to 1 mg/kg/day prednisone or equivalent. In some embodiments, the GC therapy is administered at a dose of up to 0.9 mg/kg/day, a dose of up to 0.8 mg/kg/day, a dose of up to 0.7 mg/kg/day, a dose of up to 0.6 mg/kg/day, a dose of up to 0.5 mg/kg/day, a dose of up to 0.4 mg/kg/day, a dose of up to 0.3 mg/kg/day, a dose of up to 0.2 mg/kg/day or dose of up to 0.1 mg/kg/day prednisone or equivalent.

In some embodiments, the prednisone equivalent doses are determined as shown in Table 3.

In some embodiments, the GC therapy is administered at a high dose of prednisone or equivalent. In some embodiments, the patient has been administered a high dose of GC therapy and is unlikely to respond to any treatment therapy. In some embodiments, the patient has not responded to prior therapies before administration of the anti-CD19 antibody (e.g., obexelimab).

In some embodiments, the GC therapy continues during the treatment with obexelimab. In some embodiments, the GC therapy is tapered during treatment with obexelimab. In some embodiments, the GC therapy is tapered prior to treatment with obexelimab. In some embodiments, the GC therapy is tapered to complete discontinuation. In some embodiments, obexelimab is administered in combination with a GC therapy.

### The IgG4-RD Responder Index

The IgG4-RD Responder Index (RI) was developed to help investigators assess the efficacy of treatment in a structured manner. In some embodiments, efficacy is measured using IgG4-RD RI. The IgG4-RD RI is a tool designed to detect change in disease activity and identify improvements and worsening in the same or different organ systems, compared to placebo or when compared to start of the treatment. The validation of the Index was performed by 26 physician-investigators including representatives from 6 specialties and 9 countries (Carruthers 2012, Wallace 2018). The IgG4-RD RI uses a scoring system for each organ system or site and asks a clinician to rate the extent of disease activity and damage at the time of the clinical encounter. The original IgG4-RD RI has been modified to eliminate serum IgG4 concentrations as part of the instrument, to score with improved or worsened from baseline, and to eliminate lymph node involvement, and to include an assessment of damage caused by IgG4-RD in each affected organ (Carruthers 2012). The total IgG4-RD RI activity score includes the sums of the numerical values in the activity column.

In some embodiments, the physician scores each of the organ systems as 0-3 based on the previous 28 days. An urgent organ involvement (i.e., requiring urgent treatment), in some embodiments, receives double the score. An organ can be selected from, but not limited to, lymph nodes, submandibular glands, parotid glands, lacrimal glands, kidney, heart, pericardium, orbit, nasal cavity, lungs, bile ducts, salivary glands, and pancreas. In some embodiments, the organ or organ site is selected from pachymeninges, pituitary gland, orbits and lacrimal glands, salivary glands, thyroid, lymph nodes, lungs, aorta and large blood vessels, retroperitoneum, mediastinum, and mesentery, pancreas, bile duct and liver, kidney, skin, and other sclerosis/mass formation. In some embodiments, the organ or organ site is selected from pachymeninges, pituitary gland, orbits, lacrimal glands, salivary glands, lymph nodes, lung, aorta, retroperitoneum, pancreas, bile duct, liver, kidney, and skin.

At each subsequent assessment, the physician enters a 0-3 score after the organ/site listed with,
0=Normal or resolved
1=Improved but still present since the previous assessment or improved from initial assessment with
no change from previous assessment
2=Disease activity unchanged from previous assessment
Once an organ or site has improved or worsened, the score of 2 will no longer be used for that organ/site, i.e., an improved organ/system will maintain a score of 1 until resolution (score as 0) or worsening (score as 3).
No change from previous assessment will in some embodiments refer to disease manifestations that require follow-up imaging to assess accurately, i.e., a disease manifestation only assessable by imaging will be assumed to be unchanged until such time that repeat imaging demonstrates improvement, worsening, or resolution.
3=Worsened or new disease manifestation since the last assessment and requirement for additional therapy (intent to treat).

The compiled score gives a snapshot of the disease activity at that point in time compared to the previous visit. The proportion of patients achieving complete remission by Week 52, defined as an IgG4-RD RI score of 0. In some embodiments, the patient achieves complete remission following administration of obexelimab. In some embodiments, complete remission is defined as an IgG4-RD RI score of 0, no AC determined flare, and no treatment for flare, by Week 52.

In some embodiments, the patient achieves complete remission following administration of obexelimab, wherein complete remission comprises an IgG4-RD RI score of 0. In some embodiments, the patient achieves complete remission following administration of obexelimab, wherein complete remission comprises an IgG4-RD RI score of 0, no AC determined flare, and no treatment for flare. In some embodiments, the patient achieves complete remission following administration of obexelimab, wherein complete remission comprises an IgG4-RD RI score of 0 and no treatment for flare.

In some embodiments, the IgG4-RD RI includes the subject's serum IgG4 concentration. In some embodiments, the IgG4-RD RI does not include the subject's serum IgG4 concentrations.

In one aspect, the present invention provides a method of treating IgG4-related disease (IgG4-RD), comprising administering obexelimab subcutaneously to a human patient at a dose of 250 mg once a week. In some embodiments, the method further comprises determining IgG4-RD RI. In some embodiments, IgG4-RD RI is determined prior to treatment with obexelimab (e.g., baseline). In some embodiments, IgG4-RD RI is determined following administration of obexelimab. In some embodiments, IgG4-RD RI is maintained relative to baseline. In some embodiments, IgG4-RD RI is reduced relative to baseline, wherein baseline is the IgG4-RD RI score prior to initiation of treatment.

In some embodiments, obexelimab is administered to a subject to improve or prevent worsening of signs or symptoms of IgG4-RD. In some embodiments, improving or preventing worsening of signs or symptoms of IgG4-RD is measured using IgG4-RD RI.

In some embodiments, the IgG4-RD-RI score ranges from 0-30. In some embodiments, the IgG4-RD-RI score ranges from 0-5. In some embodiments, the IgG4-RD-RI score ranges from 0-10. In some embodiments, the IgG4-RD-RI score ranges from 0-15. In some embodiments, the IgG4-RD-RI score ranges from 0-20. In some embodiments, the IgG4-RD-RI score ranges from 0-25.

In some embodiments, the IgG4-RD-RI score is decreased by at 1, 2, 3, 4, 5, 6, 7, 8, 9, 10, 11, 12, 13, 14, 15, 16, 17, 18, 19, 20, 21, 22, 23, 24, 25, 26, 26, 28 relative to baseline. In some embodiments, the IgG4-RD-RI score is decreased by 1-5, 2-5, 3-5, 4-5, relative to baseline.

In some embodiments, the IgG4-RD-RI score is decreased by at least 1 relative to baseline. In some embodiments, the IgG4-RD-RI score is decreased by at least 2 relative to baseline. In some embodiments, the IgG4-RD-RI score is decreased by at least 3 relative to baseline. In some embodiments, the IgG4-RD-RI score is decreased by at least 4 relative to baseline. In some embodiments, the IgG4-RD-RI score is decreased by at least 5 relative to baseline. In some embodiments, the IgG4-RD-RI score is decreased by at least 6 relative to baseline. In some embodiments, the IgG4-RD-RI score is decreased by at least 7 relative to baseline. In some embodiments, the IgG4-RD-RI score is decreased by at least 8 relative to baseline. In some embodiments, the IgG4-RD-RI score is decreased by at least 9 relative to baseline. In some embodiments, the IgG4-RD-RI score is decreased by at least 10 relative to baseline. In some embodiments, the IgG4-RD-RI score is decreased by at least 11 relative to baseline. In some embodiments, the IgG4-RD-RI score is decreased by at least 12 relative to baseline. In some embodiments, the IgG4-RD-RI score is decreased by at least 13 relative to baseline. In some embodiments, the IgG4-RD-RI score is decreased by at least 14 relative to baseline. In some embodiments, the IgG4-RD-RI score is decreased by at least 15 relative to baseline. In some embodiments, the IgG4-RD-RI score is decreased by at least 16 relative to baseline. In some embodiments, the IgG4-RD-RI score is decreased by at least 17 relative to baseline. In some embodiments, the IgG4-RD-RI score is decreased by at least 18 relative to baseline. In some embodiments, the IgG4-RD-RI score is decreased by at least 19 relative to baseline. In some embodiments, the IgG4-RD-RI score is decreased by at least 20 relative to baseline. In some embodiments, the IgG4-RD-RI score is reduced to 0.

In some embodiments, the IgG4-RD-RI score is decreased by at least 2 relative to baseline within 1 week following administration of obexelimab. In some embodiments, the IgG4-RD-RI score is decreased by at least 2 relative to baseline within 2 weeks following administration of obexelimab. In some embodiments, the IgG4-RD-RI score is decreased by at least 2 relative to baseline within 3 weeks following administration of obexelimab. In some embodiments, the IgG4-RD-RI score is decreased by at least 2 relative to baseline within 4 weeks following administration of obexelimab. In some embodiments, the IgG4-RD-RI score is decreased by at least 2 relative to baseline within 5 weeks following administration of obexelimab. In some embodiments, the IgG4-RD-RI score is decreased by at least 2 relative to baseline within 6 weeks following administration of obexelimab. In some embodiments, the IgG4-RD-RI score is decreased by at least 2 relative to baseline within 7 weeks following administration of obexelimab. In some embodiments, the IgG4-RD-RI score is decreased by at least 2 relative to baseline within 8 weeks following administration of obexelimab. In some embodiments, the IgG4-RD-RI score is decreased by at least 2 relative to baseline within 9 weeks following administration of obexelimab. In some embodiments, the IgG4-RD-RI score is decreased by at least 2 relative to baseline within 10 weeks following administration of obexelimab.

In some embodiments, the patient achieves complete remission by week 25 following administration of obexelimab, wherein complete remission comprises an IgG4-RD RI score of 0 and no treatment for flare. In some embodiments, the patient achieves complete remission by week 26 following administration of obexelimab, wherein complete remission comprises an IgG4-RD RI score of 0 and no treatment for flare. In some embodiments, the patient achieves complete remission by week 27 following administration of obexelimab, wherein complete remission comprises an IgG4-RD RI score of 0 and no treatment for flare. In some embodiments, the patient achieves complete remission by week 28 following administration of obexelimab, wherein complete remission comprises an IgG4-RD RI score of 0 and no treatment for flare. In some embodiments, the patient achieves complete remission by week 29 following administration of obexelimab, wherein complete remission comprises an IgG4-RD RI score of 0 and no treatment for flare. In some embodiments, the patient achieves complete remission by week 30 following administration of obexelimab, wherein complete remission comprises an IgG4-RD RI score of 0 and no treatment for flare. In some embodiments, the patient achieves complete remission by week 40 following administration of obexelimab, wherein complete remission comprises an IgG4-RD RI score of 0 and no treatment for flare. In some embodiments, the patient achieves complete remission by week 45 following administration of obexelimab, wherein complete remission comprises an IgG4-RD RI score of 0 and no treatment for flare. In some embodiments, the patient achieves complete remission by week 50 following administration of obexelimab, wherein complete remission comprises an IgG4-RD RI score of 0 and no treatment for flare.

### The Glucocorticoid Toxicity Index (GTI)

The GTI is a physician-reported measure of toxicity related to the use of GCs. It is a weighted outcome measure that scores the change in steroid toxicity over time. The index is a measure of change in corticosteroid toxicity over time and utilizes 9 domains (body mass index [BMI], glucose tolerance, blood pressure, lipids, bone density, steroid myopathy, skin toxicity, neuropsychiatric toxicity, and infection) using 31 items. The GTI was developed using multicriteria decision analysis by a group of 19 experts in corticosteroid use and aims to give a reliable measure of steroid-sparing ability of new treatment agents (Stone 2022). The GTI has been used as an important efficacy endpoint in other clinical studies (Jayne 2021, McDowell 2021). GTI is described in Stone J H, et al. Semin Arthritis Rheum. 2022 August; 55:152010. PMID: 35486995, which is hereby incorporated by reference in their entirety.

In one aspect, the present invention provides a method of treating IgG4-related disease (IgG4-RD), comprising administering obexelimab subcutaneously to a human patient at a dose of 250 mg once a week. In embodiments, the subcutaneous administration of obexelimab achieves a change in glucocorticoid-associated toxicity as measured by the GTI score.

In some embodiments, the method of treating IgG4-related disease (IgG4-RD) comprises determining toxicity associated with corticosteroid use. In some embodiments, toxicity associated with corticosteroid use is determined prior to administration of obexelimab. In some embodiments, toxicity associated with corticosteroid use is determined following administration of obexelimab. In some embodiments, toxicity associated with corticosteroid use is reduced following administration of obexelimab.

In some embodiments, toxicity associated with corticosteroid use is determined using the GTI. In some embodiments, the GTI score has a range of 346 to +439. In some embodiments, the GTI score demonstrates worsening changes of GTI corresponding to ≥10 points, ≥20 points, and ≥30 points. In some embodiments, GTI score is reduced following administration of obexelimab. In some embodiments, GTI score is reduced by at least 10 following administration of obexelimab. In some embodiments, GTI score is reduced by at least 15 following administration of obexelimab. In some embodiments, GTI score is reduced by at least 20 following administration of obexelimab. In some embodiments, GTI score is reduced by at least 25 following administration of obexelimab. In some embodiments, GTI score is reduced by at least 30 following administration of obexelimab. In some embodiments, GTI score is reduced by at least 35 following administration of obexelimab. In some embodiments, GTI score is reduced by at least 40 following administration of obexelimab. In some embodiments, GTI score is reduced by at least 45 following administration of obexelimab.

### GTI Score Factors

In some embodiments, the GTI score is measured by change in BMI by at least −5 BMI units or greater than about 5 BMI units. In some embodiments, the GTI score is measured by change in Glucose Metabolism by improvement in HbA1c and/or decrease in medication or increase in HbA1c and/or increase in medication. In some embodiments, the GTI score is measured by change in blood pressure by improvement in blood pressure and/or decrease in medication or increase in blood pressure and/or increase in medication. In some embodiments, the GTI score is measured by change in hyperlipidemia as measured by decrease in LDL and/or decrease in medication or increase in hyperlipidemia as measured by decrease in LDL and/or increase in medication. In some embodiments, the GTI score is measured by change in steroid myopathy as measured by moderate weakness to no weakness with or without functional limitation or none to mild weakness with or without functional limitation. In some embodiments, the GTI score is measured by skin steroid-related toxicity as measured by decrease or increase in skin steroid toxicity. In some embodiments, the GTI score is measured by neuropsychiatric (NP) steroid related symptoms as measured by decrease or increase in decrease in NP toxicity. In some embodiments, the GTI score is measured by infection as measured by oral and/or vaginal candidiasis and/or other grade 3, 4, or 5 infections.

The GTI is measured at baseline and then every three or six months, a period known as the GTI interval. The GTI is scored by comparing the domain values at the start of each GTI interval to the scores at the end of each GTI interval. Both the CWS and the AIS are calculated for each GTI interval, and then the interval scores are summed.

### EXAMPLES

### Example 1: Patient Selection and Inclusion Criteria

This Example describes the selection of patients with active IgG4-RD for treatment with CD19 antibody. In a 20 patient Phase II, open-label, single-arm clinical study (XmAb5871-03) in patients with active IgG4-RD, obexelimab demonstrated strong and rapid improvement in disease activity. This study included up to 4 weeks of screening, followed by 12 doses administered every 14 days (24 weeks), plus 6 weeks of follow-up. Fourteen out of 15 patients (93%) treated with obexelimab 5 mg/kg IV Q14 days achieved a ≥2 decrease in IgG4-RD Responder Index (RI), at some point during the study, and all 14 patients had decreases of 5 points or more. Notably, 1 patient had a baseline IgG4-RD RI score of 2, but still had a 50% decrease in the RI score (RI=1) by the end of the study.

Taken together, the role of CD19+ B cells in the pathogenesis of IgG4-RD, the observed effects of B-cell depletion in previous studies in IgG4-RD (Khosroshahi 2010), and the strong efficacy and safety observed in the Phase 2 IgG4-RD study with obexelimab provide a strong rationale for the continued development of obexelimab in a pivotal, double-blinded, randomized study in IgG4-RD.

The study consists of a screening period (Day −28 to Day −1) and a 52-week Randomized Control Period (RCP), during which obexelimab or placebo will be administered as SC injections every 7 days (52 doses) followed by an open label extension period. Eligible patients will meet the 2019 American College of Rheumatology (ACR)/European League Against Rheumatism (EULAR) classification criteria for IgG4-RD and must have active IgG4-RD signs/symptoms (i.e., flare) that require the initiation of glucocorticoid (GC) therapy or the increase in background long-term GC therapy. The primary endpoint is Time to IgG4-RD flare (TDF). Time to IgG4-RD flare (TDF) is the reappearance of previous signs/symptoms or appearance of new signs/symptoms of IgG4-RD from randomization to Week 52.

All patients will receive ≥3 weeks and up to a maximum of 6 weeks of GC treatment at a dose of 20 to 60 mg/day prednisone equivalent prior to randomization. The required GC therapy will be either newly initiated or an increase in long-term GC therapy (i.e., patient was previously on a dose of s; 10 mg/day prednisone equivalent). The exact dose and taper schedule, during the screening period, prior to randomization, will be at the discretion of the investigator, preferably over 0-8 weeks. On the day of randomization, patients must be at a dose of 20 mg/day prednisone equivalent and begin a protocol-specified taper to discontinuation by Week 8.

At the Day 1 visit, eligible patients will be stratified based on number of organs affected (1 or >1) and whether or not they have newly diagnosed vs reoccurring disease, because the risk of reoccurrence is likely to differ between them. Patients will be randomized 1:1 to receive obexelimab or placebo and the first SC dose of obexelimab/placebo will be administrated at the trial site. Table 2 shows the dosage and formulation of obexelimab. All patients will be observed for at least 2 hours, during which time safety assessments will be performed. Patients will be evaluated for disease activity and AEs every week.

### Placebo

Placebo without active substance will be supplied also as a solution for SC injection. The placebo formulation is: 2.35 mg/mL sodium acetate trihydrate, 0.17 mg/mL acetic acid (at density 1.053 g/mL), 30 mg/mL L-proline, 0.1 mg/mL polysorbate 80, 115 mg/mL Dextran-40 at pH 5.5.

The SC formulation of the placebo is a sterile liquid product supplied in single-use glass vials. Each 2-mL glass vial is filled with 1.2 mL of placebo. The single-use glass vial is masked to make it indistinguishable from obexelimab. Table 6 shows the dosage regimen and formulation of obexelimab.

TABLE 6
Investigational Product: obexelimab and dosage

| Study Investigational Product: | | obexelimab | |
|---|---|---|---|
| Dosage formulation: | | Solution for injection | |
| Route of administration: | | Subcutaneous | |
| Formulation: | | Formulation: 125 mg/mL | |
| | | obexelimab, 2.35 mg/mL | |
| | | sodium acetate trihydrate, | |
| | | 0.17 mg/mL acetic acid | |
| | | (at density 1.053 g/mL), | |
| | | 30 mg/mL L-proline, 0.1 | |
| | | mg/mL polysorbate 80, | |
| | | pH 5.5 | |
| Unit dose strength(s)/dosage level(s): | | 125.0 (±10%) mg/mL | |
| Dosing instructions: | | 2 × 1.0 mL administered SC | |
| | | to the abdominal region | |
| Packaging: | | Single-use 2-mL glass vials, | |
| | | each filled with 1.2 mL of drug | |
| | | product or pre-filled syringe | |
| Dosage | Cₘₐₓ | | |
| regimen | (mean steady state) | AUC_{0-336 h} | Cₘᵢₙ |
| 10 mg/kg IV | 272,832 ng/ml | 18,182,275 h*ng/ml | 3,707 ng/ml |
| every 14 days | | | |
| 250 mg SC | 24,793 ng/ml | 7,374,758 h*ng/ml | 17,128 ng/ml |
| every 7 days | | | |

During the Randomized control period (RCP), patients will undergo assessments for efficacy, safety, PK, pharmacodynamics (PD), and immunogenicity at study visits specified in the Schedule of Assessments (SoA). Adverse events (AE), serious adverse events (SAE) and treatment-emergent adverse events (TEAEs) or clinically significant safety laboratory abnormalities will be evaluated.

### Inclusion Criteria

Patients are eligible to be included in the study only if all of the following criteria apply:
1. Males and females ≥18 years of age at the time of signing the informed consent
2. Patients must meet the 2019 ACR/EULAR classification criteria for IgG4-RD with a score of ≥20
3. Patients must have active IgG4-RD signs/symptoms (i.e., flare) that require, as assessed by the investigator, the initiation of GC therapy or the increase in background long-term GC therapy (if previously on stable dose of <10 mg/day prednisone equivalent)
4. Total duration of GC treatment prior to randomization must be ≥3 weeks and a maximum of 6 weeks at a dose of 20 to 60 mg/day
5. A female patient is eligible to participate if she is not pregnant (see Appendix 4), not breastfeeding, and at least 1 of the following conditions applies:
   a. Not a woman of childbearing potential (WOCBP) OR
   b. A WOCBP who agrees to follow the contraceptive guidance after the last administration of study drug
6. A male patient must:
   a. Agree to (i) abstain from intercourse or (ii) use contraception for at least 1 month (i.e., approximately 5 half-lives) after the last dose of IP, or (iii) be surgically sterile for the duration of the study and
   b. Agree to refrain from donating sperm during this period for at least 1 month (i.e., approximately 5 half-lives) after the last dose of IP
7. A WOCBP must have a negative serum pregnancy test at screening and a negative urine test prior to the first dose of study drug and at all timepoints specified in the SoA.

### Exclusion Criteria

Patients are excluded from the study if any of the following criteria apply:
1. Any exclusion criteria listed in the ACR/EULAR IgG4-RD Classification Criteria
2. Patients with disease in only 1 organ system whose primary manifestation is fibrosis (i.e., retroperitoneum fibrosis without aortitis, Riedel's thyroiditis, fibrosing mediastinitis, sclerosing mesenteritis involvement, etc.)
3. Has received prednisone equivalent given orally at a dose greater than 60 mg/day within the 4 weeks prior to screening or during screening.
4. Unable to tolerate 20 mg/day on Day 1.
5. Unable to taper off of GC therapy by 8 weeks post randomization.
6. Use of B cell depleting, B cell targeted, or other biologic immunomodulatory agents within the 6 months prior to randomization. Patients who received B cell targeted therapy within 6 to 12 months prior to randomization must have a B cell count that is within the laboratory reference range at screening, as measured by the central laboratory.
7. Has received a non-biologic, disease-modifying anti-rheumatological drugs or immunosuppressive agent other than GCs within the 4 weeks prior to screening
8. Has received an investigational treatment or direct medical intervention on another clinical study within 12 weeks or <5 half-lives of the investigational treatment, whichever is shorter, prior to screening.
9. Has received live vaccine or live therapeutic infectious agent within the 2 weeks prior to screening.
10. Acute hepatitis B infection (hepatitis B surface antigen-positive), active hepatitis C virus, or HIV infection. A positive test for hepatitis B is detection of either (a) hepatitis B surface antigen or (b) hepatitis B core antibody; and in Japan only (c) hepatitis B surface antibody.
11. Evidence of active tuberculosis (TB) or at high risk for TB based on the following:
   a. History of active TB or latent TB, unless completion of treatment according to local guidelines is documented.
   b. Positive interferon-gamma release assay results at screening, unless treatment is documented.
   c. Signs of symptoms that could represent active TB.
   d. Chest radiograph, computed tomography (CT), or magnetic resonance imaging (MRI) that suggests possible diagnosis of TB.
12. History or evidence of a clinically unstable/uncontrolled disorder, condition, or disease (including, but not limited to, cardiopulmonary, oncologic, renal, hepatic, metabolic, hematologic, psychiatric, active infection) other than IgG4-RD that, in the opinion of the investigator, would pose a risk to patient safety or interfere with the study evaluation, procedures, or completion.
13. Malignancy within 5 years (except successfully treated in situ cervical cancer, resected squamous cell or basal cell carcinoma of the skin, breast cancer with no recurrence ≥5 years following therapy, or prostate cancer with no recurrence ≥3 years following prostatectomy)
14. Any known allergy to monoclonal antibody therapy.
15. Hematology or clinical chemistry parameters that meet any of the following criteria at screening:
   a. White blood cell count <2.5×10³/μL
   b. Absolute neutrophil count <1.0×10³/μL
   c. Elevated serum creatinine >2.5× upper limit of normal (ULN) OR estimated creatinine
   clearance <40 L/min calculated by the Cockrock-Gault formula at screening
   d. Hemoglobin <10 g/dL
   e. Platelet count <75×10³/μL
16. Abnormal liver function tests meeting any of the following criteria:
   a. In the absence of hepatobiliary activity:
      i. Alanine aminotransferase (ALT) >2×ULN
      ii. Aspartate aminotransferase (AST) >2×ULN
      iii. Total bilirubin ≥2×ULN
   b. In the presence of hepatobiliary activity:
      i. ALT ≥10×ULN
      ii. AST ≥10×ULN
      iii. Total bilirubin ≥5×ULN

Patients receiving obexelimab experience significantly reduced flare occurrences, when compared to patients receiving placebo.

### Example 2: Prior Glucocorticoid Therapy

Prior to randomization, all patients will receive ≥3 weeks and up to a maximum of 6 weeks of GC therapy at a dose of 20 to 60 mg/day prednisone equivalent (Table 3). GC therapy can be either newly initiated or an increase in long-term GC therapy (e.g., increase from a previously stable dose of <10 mg/day prednisone equivalent).

TABLE 3
Prednisone Equivalence

| Corticosteroid | Dose equivalent to 10 mg prednisone |
|---|---|
| Hydrocortisone | 40 mg |
| Prednisolone | 10 mg |
| Triamcinolone | 8 mg |
| Methylprednisolone | 8 mg |
| Dexamethasone | 1.5 mg |
| Betamethasone | 1.2 mg |

The exact dose and schedule prior to randomization, which may include a taper schedule, if deemed necessary, are at the discretion of the investigator. However, patients must be at a dose of 20 mg/day prednisone equivalent on the day of randomization and receive that dose of GC therapy on Day 1. Patients will be tapered to discontinuation over 8 weeks, with dose reductions of 5 mg equivalent every 2 weeks (Table 4).

TABLE 4
Glucocorticoid Taper*

| Weeks Dose of oral | Dose of oral |
|---|---|
| prednisone (or equivalent) | prednisone (or equivalent) |
| Week 0 (Day 1) through end of Week 1 | 20 mg/day |
| Week 2 through end of Week 3 | 15 mg/day |
| Week 4 through end of Week 5 | 10 mg/day |
| Week 6 through end of Week 7 | 5 mg/day |

*Patients must be at a dose of 20 mg/day prednisone equivalent on Week 0 (Day 1) and will undergo a required GC taper to discontinuation over 8 weeks, with dose reductions of 5 mg equivalent every 2 weeks.

### Example 3: Efficacy Analyses and Endpoints

The primary efficacy endpoint (primary analysis) is time to IgG4-RD flare (TDF), which is the reappearance of previous signs/symptoms or appearance of new signs/symptoms of IgG4-RD from randomization to Week 52 that, require initiation of rescue therapy. The proportion of patients achieving complete remission by Week 52, is IgG4-RD RI score of 0, and involves no flare, and no treatment for flare, by Week 52.

The date of IgG4-RD flare is defined as the date of initiation of any flare treatment (new GC treatment, other immunotherapy, or interventional procedure) deemed necessary for the disease flare. A hazard ratio of 0.37 is used as a threshold for trial success. The hazard ratio of 0.37 corresponds to 35% of patients expected to have a disease flare (and 30 such patients required) by 52 weeks in the placebo group and 15% of patients expected to have a disease flare (13 such patients required) in the obexelimab group by 52 weeks.

Extensions of Kaplan-Meier (K-M) estimation (Aalen and Johansen 1978) and the log-rank test (Gray 1988) will be used for an intent-to-treat comparison of obexelimab and placebo across the entire observed follow-up period. The extensions accommodate adjustment for stratification and competing risk (if required), where the strata are 1 organ vs ≥1 organ and newly diagnosed vs reoccurring and competing risks are of four possible types:
1. death possibly partly due to IgG4-RD,
2. drop-out due to perceived lack of efficacy, and
3. drop-out due to side-effects or intolerance of study drug
4. due to the adjudication committee (AC) not agreeing with initiation of rescue therapy by the Investigator.

The estimation of flare incidence is limited to less than 100% in the presence of competing risk, so the competing risks need to reflect realistic scenarios which are alternative to and occurring before detecting flare, and are hopefully not frequent. Two of the possible sources of competing risk, related death and drop-out due to lack of efficacy, are very unlikely to occur before identification of IgG4-RD flare, but may occur before flare in the event that data identifying the flare rescue treatment is missing. In an actual clinical setting, such events may occur before identification of flare due to a lesser degree of patient monitoring than occurs during a clinical study.

If there are frequent competing risk events, or frequent missing dates requiring interval censoring, tipping-point analysis will be employed to explore the robustness of the analysis. For example, if the primary result is statistically significant, the conditions under which the result could become non-significant would be explored. For interval censoring (date of flare rescue treatment unknown but we have a minimum date and a maximum date), this would entail placing different numbers of such results at their minimum date for obexelimab arm, and maximum date for placebo arm. For competing risks, this would entail re-assigning different numbers of such events to be flare events for obexelimab arm and right censored for placebo arm.

In addition to the primary analysis, a sensitivity analysis using a proportional hazards model will be performed to evaluate treatment effect adjusted for impactful prognostic factors and baseline covariates. The model will use time-to-disease flare (TDF) as the response variable, with treatment arm, stratification factors, subgroup variables and continuous variables (e.g., age) as covariates. Table 5 summarizes the secondary end points.

TABLE 5
Summary of Secondary Efficacy Endpoints and Analyses

| Endpoint | Statistical analysis methods |
|---|---|
| Time to IgG4-RD flare | Analysis will be the same as primary analysis |
| (TDF) that requires | |
| initiation of rescue | |
| therapy, as determined | |
| by the investigator | |
| GC rescue therapy use | The duration and cumulative dose of IgG4-RD |
| | GC rescue therapy use from randomization to |
| | Week 52 will be summarized and listed. Both |
| | duration and cumulative dose may be estimated |
| | using time-to-event methods, adjusting for |
| | censoring and competing risk, with unexposed |
| | patients who complete 52 weeks being |
| | censored at duration and dose zero. |
| Proportion of patients | Pearson's chi-square test with multiple |
| achieving complete | imputation for missing data. |
| remission | |
| Incidence of IgG4-RD | Multiple flare events per patients will be taken |
| AC determined flare | into account and an appropriate estimator of |
| events, | cumulative incidence over time which may |
| | be adjusted for censoring and competing risk |
| | (e.g., Aalen-Johansen estimator) will be |
| | computed. These are annualized rates at 52 |
| | weeks. |
| expressed as annualized | A ratio (obexelimab over placebo) of such |
| flare rate | incidence rates at Week 52 may be computed |
| | for comparison, with a confidence interval. |
| | The number and frequency of flares will also |
| | be shown by visit. |
| IgG4-RD RI | Treatment comparison and descriptive statistics |
| | of the improvement over baseline as measured |
| | by the IgG4-RD RI will be shown at all visits |
| GTI | Treatment comparison and descriptive statistics |
| | for changes from baseline in GTI at all visits. |
| | Proportions of patients, by treatment group with |
| | worsening changes of GTI corresponding to ≥10 |
| | points, ≥20 points, and ≥30 points will also be |
| | examined. |

### Example 4: PK/PD Analysis of SC Administered Obexelimab

This example describes exemplary PK/PD analysis of SC administered obexelimab. Simulation experiments demonstrated that 250 mg obexelimab administered SC every 7 days dose of achieves maximum suppression of absolute B cell count of approximately 50% of baseline and 100% receptor occupancy of CD19 during the entire dosing interval. The PK and PD simulation of 250 mg SC every 7 days supports sufficient safety coverage (lower concentration maximum [Cmax] and area under the curve [AUC]) while maintaining higher trough concentrations for target engagement when compared with PK from previous studies of IV administration. Compared with 10 mg/kg IV every 14 days, the mean steady-state Cmax and AUC (normalized to 336 hours) for 250 mg SC every 7 days is 11-fold and 2.5-fold lower, respectively. All mAb therapeutics given by the intravenous route are associated with the risk of both non-allergic (cytokine release syndrome) and allergic (hypersensitivity) infusion-related reactions.

However, the mean steady-state trough concentration of 250 mg SC every 7 days is 4.6-fold higher, which is likely to provide better maintenance of target engagement and maximum PD effects. This data suggests that treatment of IgG4-RD with obexelimab will provide reduction in the risk and frequency of disease flares.

### Example 5: PK/PD Simulation Models Validate the Obexelimab Dosing in Patients with Immunoglobulin G4-Related Disease (IgG4-RD)

This Example illustrates the validation of suitable Obexelimab doses for treating patients with immunoglobulin G4-related disease (IgG4-RD) using PK/PD.

A population PK/PD model, which described obexelimab plasma PK and the exposure-response (E-R) relationship of plasma obexelimab concentrations and absolute B cell (ABC) count or CD19 receptor occupancy (RO) following single and multiple intravenous (IV) or subcutaneous (SC) administration to healthy subjects, and patients with RA or IgG4-RD, was established based on 4 studies (two Phase I studies and two Phase 2 studies). A PK model was a two-compartmental model with first-order absorption for SC administration and first order elimination. Two covariates were included in the PK model-dose and disease status.
Dose was included as a covariate on the scaling factor in the PK model described the nonlinear PK of obexelimab observed at low doses using as an Eₘₐₓ model.
Disease status was included as a covariate on obexelimab elimination rate from the central compartment. The final ABC count PK/PD model was an indirect response model.

The increased ABC disappearance rate (Kₒᵤₜ) by obexelimab was described by a sigmoidal Eₘₐₓ function. The CD19 RO PK/PD model was a direct response model with a sigmoidal Eₘₐₓ function. Table 6 lists the parameters estimated for the PK, ABC PK/PD, and CD19 RO PK/PD model.

TABLE 6
parameters estimated for the PK/PD model.

| | | PK/PD model | | | |
|---|---|---|---|---|---|
| PK model | | of ABC count | | PK/PD model of CD19 | |
| Parameter | Estimates | | 90% CI | receptor occupancy | |
| kel (hr⁻¹) | 0.0111 | kel (hr⁻¹) | FIXED | kel (hr⁻¹) | FIXED |
| V2 (L) | 11.8562 | V2 (L) | FIXED | V2 (L) | FIXED |
| Q (L/hr) | 0.0091 | Q (L/hr) | FIXED | Q (L/hr) | FIXED |
| V3 (L) | 1.4925 | V3 (L) | FIXED | V3 (L) | FIXED |
| ka (hr⁻¹) | 0.0101 | ka (hr⁻¹) | FIXED | ka (hr 1) | FIXED |
| F1 | 0.6093 | F1 | FIXED | F1 | FIXED |
| Vmax on sc | 0.6610 | Vmax on sc | FIXED | Vmax on sc | FIXED |
| Km on sc | 4.0945 | Km on sc | FIXED | Km on sc | FIXED |
| (mg) | | (mg) | | (mg) | |
| RA on kel | 0.1465 | RA on kel | FIXED | RA on kel | FIXED |
| — | — | Kin | 0.03295 | Emax | 0.984108 |
| — | — | Kout0 | 0.035907 | EC50 (ng/ml) | 74.4611 |
| — | — | EC50 (ng/ml) | 98.5764 | γ | 9.69832 |
| — | — | Emax | 0.80907 | — | — |
| IIV on kel | 0.072849 | IIV on kel | 0.06939 | — | — |
| IIV on V2 | 0.115092 | IIV on V2 | 0.118322 | — | — |
| IIV on ka | 0.141428 | IIV on ka | 0.14004 | — | — |
| — | — | IIV on Kout0 | 0.101303 | — | — |
| — | — | IIV on Emax | 0.199613 | — | — |
| Proportional | 0.3587 | Proportional | 0.342991 | Proportional | 0.328875 |
| residual error | | residual error | | residual error | |
| — | — | on PK | | on PK | |
| | | Proportional | 0.233979 | Proportional | 0.430542 |
| | | residual error | | residual error | |
| | | on PD | | on PD | |
| — | — | — | — | — | |

IIV = interindividual variability;
PK model:
kel = elimination rate constant,
V2 = distribution volume of plasma compartment,
Q = intercompartmental clearance,
V3 = distribution volume of peripheral compartment,
ka = absorption rate constant for SC dosing,
F1 = bioavailability of SC dose,
Vmax = proportional constant between IV dose and scaling factor for IV dose,
sc = dose scaling factor for IV administration,
Km = IV dose achieving 50% saturation of the binding sites,
RA on kel = Rheumatoid arthritis patients in study 2 PK/PD model of ABC count:
Kin = zero order rate constant describing ABC counts formation,
Kout0 = first order rate constant describing disappearance of ABC count,
EC₅₀ = plasmas obexelimab concentration achieving half the maximum ABC count reduction,
Emax = maximum effect associated with plasma obexelimab concentration PK/PD model of CD19 RO:
Emax = maximum fractional change from baseline for CD19 RO,
EC₅₀ = plasma obexelimab concentration achieving half the maximum CD19 RO reduction = sigmoidal parameter which controls the steepness of the E-R relationship

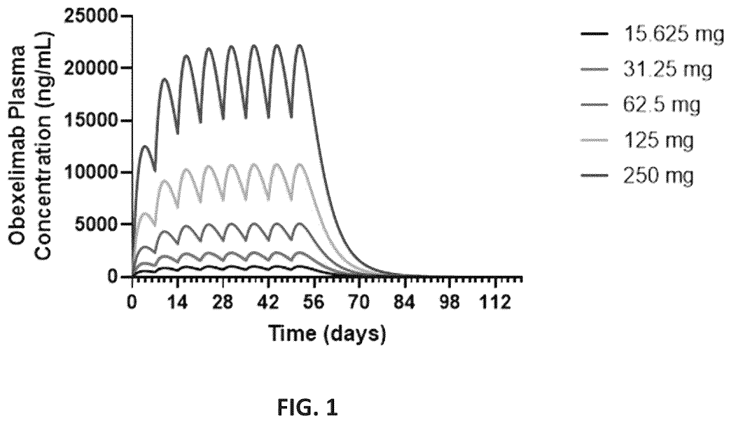

The population PK/PD model was used to simulate obexelimab PK exposure values and change in ABC count and in CD19 RO following various SC doses administration. Table 7 lists the predicted 10th, 25th, 50th, 75th and 90th percentiles of minimum plasma obexelimab concertation (Cmin), maximum plasma obexelimab concentration (Cmax), average plasma obexelimab concentration (Cavg) and area under the plasma concentration-time curve (AUC) at the first week and steady state following various QW SC doses administrations. FIGS. 1-3 illustrates the plasma obexelimab concentration-time profiles, ABC relative to baseline and CD19 RO with time.

Based on the simulated 90th percentile for plasma obexelimab C_{max,ss}, 90% of the subjects receiving 250 mg of obexelimab QW would have a plasma obexelimab concertation less than 39265 ng/mL, which is 6.9-fold less than the observed mean Cmax (272,832 ng/mL) at 10 mg/kg IV from Study XmAb5871-02.

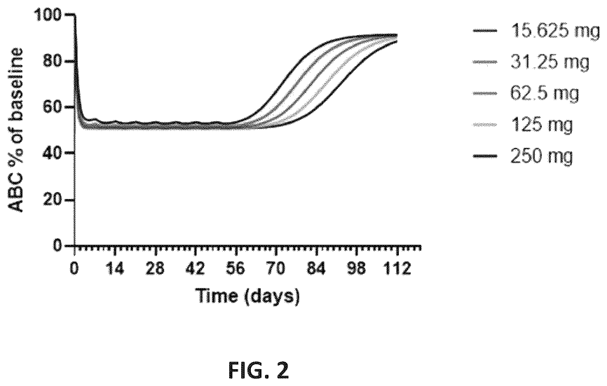

Based on the simulated 10th percentile for plasma obexelimab C_{min,ss}, 90% of the subjects receiving 250 mg of obexelimab QW would have a plasma obexelimab concertation greater than 8376 ng/mL (FIG. 1), which is at least 85.0-fold greater than the EC50 and 9.44-fold greater than the EC90 values of ABC count. The decrease of ABC count with plasma obexelimab exposure was rapid and achieves a maximum decrease of approximately 50% of baseline ABC count values. The magnitude of obexelimab impact on ABC count was similar for 15.625, 31.25, 62.5, 125 and 250 mg administrated SC QW during dosing interval with different recovery phase after stopping dosing. Once obexelimab 250 mg weekly dose administration was discontinued, approximately 10 weeks was required for ABC count to return to baseline levels (FIG. 2).

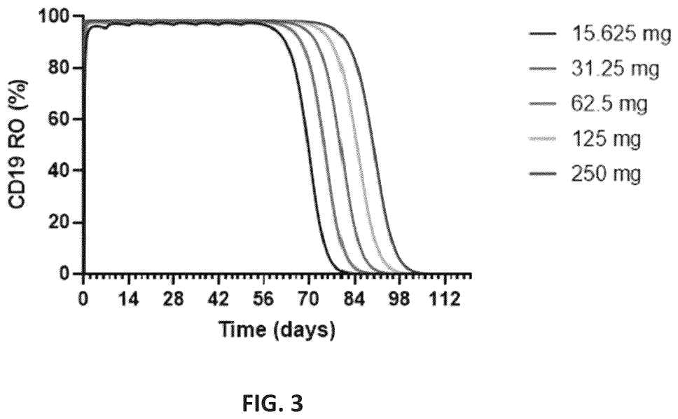

Based on the simulated 10th percentile for plasma obexelimab C_{min,ss}, 90% of the subjects receiving 250 mg of obexelimab QW would have a plasma Obexelimab concertation greater than 8376 ng/mL (FIG. 1), which is at least 112-fold greater than the EC50 and 37.1-fold greater than the EC90 values of CD19 RO. The increase CD19 RO with plasma obexelimab exposure was rapid and achieves a RO of approximate 100%. The magnitude of obexelimab impact on CD19 RO was similar for 15.625, 31.25, 62.5, 125 and 250 mg administrated SC QW during dosing interval with different recovery time back to bassline after stopping dosing. Following discontinuation of a 250 mg obexelimab weekly dosing, approximately 8 weeks is required for CD19 RO to return to baseline levels (FIG. 3).

TABLE 7
Population PK Model Prediction of First Week and
Steady-State Plasma Obexelimab Concentrations
Following 125 and 250 mg QW SC Doses Administrations

| | | QW | | | | | | |
|---|---|---|---|---|---|---|---|---|
| | PK | Dose | PK Parameter summary | | | | | |
| Time | Parameter | (mg) | mean | 10^{th} | 25^{th} | median | 75^{th} | 90^{th} |
| First | Cₘᵢₙ | 125 | 5084 | 2806 | 3561 | 4712 | 6219 | 7844 |
| week | (ng/ml) | 250 | 10468 | 5777 | 7332 | 9703 | 12804 | 16151 |
| | C_{avg} | 125 | 5226 | 2779 | 3669 | 4829 | 6327 | 8241 |
| | (ng/mL) | 250 | 10761 | 5722 | 7555 | 9943 | 13028 | 16969 |
| | Cmax | 125 | 6388 | 3438 | 4538 | 5926 | 7702 | 9981 |
| | (ng/mL) | 250 | 13154 | 7079 | 9344 | 12202 | 15857 | 20552 |
| | AUC_{-0-168 h} | 125 | 878013 | 466893 | 616409 | 811300 | 1063007 | 1384542 |
| | (ng/ml*h) | 250 | 1807824 | 961331 | 1269183 | 1670461 | 2188726 | 2850760 |
| Steady | Cₘᵢₙ | 125 | 8318 | 4068 | 5382 | 7367 | 10334 | 13781 |
| state | (ng/mL) | 250 | 17128 | 8376 | 11080 | 15170 | 21278 | 28376 |
| | C_{avg} | 125 | 10660 | 5658 | 7288 | 9679 | 13051 | 17072 |
| | (ng/ml) | 250 | 21949 | 11649 | 15005 | 19928 | 26872 | 35151 |
| | Cₘₐₓ | 125 | 12041 | 6598 | 8414 | 11042 | 14579 | 19070 |
| | (ng/ml) | 250 | 24793 | 13585 | 17323 | 22736 | 30018 | 39265 |
| | AUCₜₐᵤ | 125 | 1790865 | 950494 | 1224337 | 1626011 | 2192605 | 2868088 |
| | (ng/ml*h) | 250 | 3687379 | 1957067 | 2520902 | 3347954 | 4514556 | 5905379 |

Cmin: minimal plasma concertation;
Cavg: average plasma concentration;
Cmax; maximum plasma concentration;
AUC0-168 h: the area under the plasma concentration-time curve from time 0 to 168 hours;
Cmin, ss: minimal plasmaconcentration at steady state;
Cavg, ss: average plasma concentration at steady state;
Cmax, ss; maximum plasma concentration at steady state;
AUCtau: the area under the plasma concentration-time curve over a dosing interval at steady state (tau = 168 hours)

From the PK/PD simulation, it was observed that higher Cₘᵢₙ and comparable AUC was achieved with 250 mg SC QW (See Table 8). In addition, compared with 250 mg SC Q2W, the weekly 250 mg SC dose can provide higher AUC and Cₘₐₓ. Meanwhile, sufficient safety margins were maintained based on the data from both clinical and nonclinical studies.

TABLE 8
Summary of Subcutaneous delivery of Obexelimab administered
Q2W, as compared to QW and Safety Margin

| | Cₘₐₓ | AUC | Safety Margin based | | Safety Margin | |
|---|---|---|---|---|---|---|
| | (μg/ | (μg/ | on nonclinical data^{a} | | based on clinical data^{b} | |
| | mL) | ml*h) | For Cₘₐₓ | For AUC | For Cₘₐₓ | For AUC |
| 250 mg SC | 24.8 | 7,375 | 238 | 339 | 11 | 2.5 |
| QW | | | | | | |
| 250 mg SC | 14.7 | 3095 | 401 | 808 | 19 | 5.9 |
| Q2W | | | | | | |

^{a}based on Weekly GLP 12-week study: Cmax of 5,900 μg/mL, and AUC of 52,000 μg/mL* day or 1,250,000 μg/mL*h (averaged from male and female monkeys) at the NOAEL of 100 mg/kg IV QW (AUC of 2,500,000 μg/mL*h if normalized to 14 days for comparison with Q2W);
^{b}based on Study XmAb5871-02: Cmax

It was observed that the subcutaneous (SC) dose achieved better PK exposures than those observed at 250 mg SC Q2W, while maintaining sufficient safety coverage (See Table 8).

1. A method of treating IgG4-related disease (IgG4-RD), comprising administering obexelimab subcutaneously to a human patient at least 18 years of age at a dose of 250 mg once a week.

2. The method of claim 1, wherein the obexelimab is administered as 2×1 mL injections or 1×2 mL injection.

3. The method of claim 1 or 2, wherein the obexelimab is administered in a liquid formulation comprising 125 mg/mL obexelimab, 2.35 mg/mL sodium acetate trihydrate, 0.17 mg/mL acetic acid, 30 mg/mL L-proline, 0.1 mg/mL polysorbate 80 at pH 5.5.

4. The method of any one of the preceding claims, wherein the human patient has received glucocorticoid (GC) therapy.

5. The method of claim 4, wherein the GC therapy is administered at a dose of 20-60 mg/day prednisone or equivalent.

6. The method of claim 4 or 5, wherein the GC therapy continues during the treatment of obexelimab.

7. The method of claim 4 or 5, wherein the GC therapy is tapered.

8. The method of claim 7, wherein the GC therapy is tapered to complete discontinuation.

9. The method of any one of claims 1-3, wherein the obexelimab is administered in combination with a GC therapy.

10. The method of claim any one of the preceding claims, wherein obexelimab is administered for a time period sufficient to improve, stabilize or reduce one or more symptoms of IgG4-RD relative to a control.

11. The method of claim 10, wherein the at least one symptom is exhibited in an organ selected from lymph nodes, submandibular glands, parotid glands, lacrimal glands, kidney, heart, pericardium, orbit, nasal cavity, lungs, liver or bile ducts, salivary glands, and pancreas.

12. The method of any one of the proceeding claims, wherein obexelimab is administered to the patient to improve clinical symptoms.

13. The method of any one of the preceding claims, wherein obexelimab is administered to the human patient during a recurrence-free period to prevent relapse.

14. The method of claim 13, wherein the recurrence-free period is determined by the days to disease flare.

15. The method of claim 14, wherein the recurrence-free period is at least 60 days, 70 days, 80 days, or 90 days from the first administration of obexelimab.

16. The method of claim 15, wherein the recurrence-free period is up to 40 weeks, up to 45 weeks, up to 50 weeks, up to 52 weeks from the first administration of obexelimab.

17. The method of any one of the proceeding claims, wherein obexelimab is administered to the patient until the patient has active IgG4-RD disease flare that requires initiation of rescue therapy.

18. The method of claim 17, wherein the rescue therapy comprises administration of GC therapy.

19. The method of any of the preceding claims, wherein the human patient is relapsed or refractory to a previous treatment for IgG4-RD.

20. The method of claim 19, wherein the human patient is relapsed or refractory to rituximab.

21. The method of any of one of the preceding claims, wherein the human patient is assessed for disease activity using the IgG4-RD responder index (RI).

22. The method of any of one of the preceding claims, wherein the human patient achieves a ≥2 points decrease in IgG4-RD RI from day 1 of administration with obexelimab.

23. The method of any of one of the preceding claims, wherein the human patient has American College of Rheumatology (ACR)/European League Against Rheumatism (EULAR) classification criteria for IgG4-RD score (IgG4-RD score) ≥20 prior to administration of obexelimab.

24. The method of any one of the preceding claims, wherein the human patient presents with IgG4-RD manifestation selected from the group consisting of IgG4-related sialadenitis (chronic sclerosing sialadenitis, Kuttner's tumour, Mikulicz's disease), IgG4-related dacryoadenitis (Mikulicz's disease), IgG4-related ophthalmic disease (idiopathic orbital inflammatory disease, orbital pseudotumor), chronic sinusitis IgG4-related hypophysitis (IgG4-related panhypophysitis, IgG4-related adenohypophysitis, IgG4-related infundibuloneurohypophysitis, autoimmune hypophysitis), IgG4-related pachymeningitis, IgG4-related leptomeningitis (idiopathic hypertrophic pachymeningitis), IgG4-related pancreatitis (Type 1 autoimmune pancreatitis, IgG4-related AIP, lymphoplasmacytic sclerosing pancreatitis, chronic pancreatitis with diffuse irregular narrowing of the main pancreatic duct), IgG4-related lung disease (Pulmonary inflammatory pseudotumour), IgG4-related pleuritis, IgG4-related hepatopathy, IgG4-related sclerosing cholangitis, IgG4-related cholecystitis, IgG4-related aortitis (inflammatory aortic aneurysm), IgG4-related periaortitis (chronic periaortitis), IgG4-related periarteritis, IgG4-related pericarditis, IgG4-related mediastinitis (fibrosing mediastinitis), IgG4-related retroperitoneal fibrosis (retroperitoneal fibrosis, Albarran-Ormond syndrome, Ormond's disease (retroperitoneal fibrosis), perirenal fasciitis, Gerota's fasciitis/syndrome, periureteritis fibrosa, sclerosing lipogranuloma, sclerosing retroperitoneal granuloma, non-specific retroperitoneal inflammation, sclerosing retroperitonitis, retroperitoneal vasculitis with perivascular fibrosis), IgG4-related mesenteritis (subtypes are: mesenteric panniculitis, mesenteric lipodystrophy and retractile mesenteritis) (sclerosing mesenteritis, systemic nodular panniculitis, liposclerosis mesenteritis, mesenteric Weber-Christian disease, mesenteric lipogranuloma, xanthogranulomatous mesenteritis), IgG4-related mastitis (sclerosing mastitis), IgG4-related kidney disease (IgG4-RKD), IgG4-related tubulointerstitial nephritis (IgG4-TIN), IgG4-related membranous glomerulonephritis (idiopathic tubulointerstitial nephritis), IgG4-related prostatitis, IgG4-related perivasal fibrosis (chronic orchialgia), IgG4-related paratesticular pseudotumor, IgG4-related epididymo-orchitis (paratesticular fibrous pseudotumor, inflammatory pseudotumor of the spermatic cord, pseudosarcomatous myofibroblastic proliferations of the spermatic cord, proliferative funiculitis, chronic proliferative periorchitis, fibromatous periorchitis, nodular periorchitis, reactive periorchitis, fibrous mesothelioma), IgG4-related lymphadenopathy, IgG4-related skin disease (angiolymphoid hyperplasia with eosinophilia, cutaneous pseudolymphoma), IgG4-related perineural disease, and IgG4-related thyroid disease (Reidel's thyroiditis), inflammatory pseudotumour, and multifocal fibrosclerosis.

25. The method of anyone of the preceding claims, wherein the human patient presents an IgG4-RD manifestation selected from the group consisting of autoimmune pancreatitis (lymphoplasmacytic scleorising pancreatitis), eosinophilic angiocentric fibrosis (affecting the orbits and upper respiratory tract), fibrosing mediastinitis, idiopathic hypertrophic pachymeningitis, idiopathic tubulointerstitial nephritis, inflammatory pseudotumour, Kuttner's tumour, Mikulicz's disease, fibrosclerosis, periaortitis, periarteritis, inflammatory aortic multifocal aneurysm, Ormond's disease (tetroperitoneal fibrosis), Riedel's thyroiditis, and sclerosing mesenteritis.


SEQUENCE LISTING (12 sequences)

SEQ ID NO: 1 - protein, 556 aa, Homo sapiens [sequence omitted]

SEQ ID NO: 2 - protein, 16 aa, synthetic construct
RSSKSLQNVNGNTYLY

SEQ ID NO: 3 - protein, 7 aa, synthetic construct
RMSNLNS

SEQ ID NO: 4 - protein, 9 aa, synthetic construct
MQHLEYPIT

SEQ ID NO: 5 - protein, 5 aa, synthetic construct
SYVMH

SEQ ID NO: 6 - protein, 14 aa, synthetic construct
WIGYINPYNDGTKY

SEQ ID NO: 7 - protein, 12 aa, synthetic construct
GTYYYGTRVFDY

SEQ ID NO: 8 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 12 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS